(19) United States
(12) Patent Application Publication
(10) Pub. No.: US 2025/0231157 A1
(43) Pub. Date: Jul. 17, 2025

(54) METHOD FOR DETERMINING THE PARTITION OF NAPHTHENIC ACIDS FROM A PRODUCTION WATER MIMICKING SYSTEM AND MIMICKING SYSTEM

(71) Applicants: PETRÓLEO BRASILEIRO S.A. – PETROBRAS, Rio de Janeiro (BR); SERVIÇO NACIONAL DE APRENDIZAGEM INDUSTRIAL-DEPARTAMENTO REGIONAL DO ESTADO DO RIO DE JANEIRO-SENAI, Rio de Janeiro (BR)

(72) Inventors: Giovanni Bressiani PEDROSO, Rio de Janeiro (BR); Leonardo Guimaraes DA SILVA, Rio de Janeiro (BR); Izadora DA SILVA SANTOS, Rio de Janeiro (BR); Antonio Augusto FIDALGO NETO, Rio de Janeiro (BR); Rogerio MESQUITA DE CARVALHO, Rio de Janeiro (BR); Leonardo Santos DE BARROS, Rio de Janeiro (BR); Marco Antonio GOMES TEIXEIRA, Niterói (BR)

(73) Assignees: PETRÓLEO BRASILEIRO S.A. – PETROBRAS, Rio de Janeiro, RJ (BR); SERVIÇO NACIONAL DE APRENDIZAGEM INDUSTRIAL-DEPARTAMENTO REGIONAL DO ESTADO DO RIO DE JANEIRO-SE, Rio de Janeiro (BR)

(21) Appl. No.: 18/926,094

(22) Filed: Oct. 24, 2024

(30) Foreign Application Priority Data
Oct. 31, 2023 (BR) 1020230227848

(51) Int. Cl. G01N 31/16; G01N 1/28; G01N 1/38; G01N 33/18

(52) U.S. Cl. CPC G01N 31/16; G01N 1/286; G01N 1/38; G01N 33/18; G01N 2001/2866

(57) ABSTRACT

The present invention relates to a method for determining the partition of naphthenic acids in a system that mimics production water, comprising: (a) mixing, in a mimicking system, an aqueous phase and an oily phase comprising acidic species, varying parameters of pH, salinity, alkalinity as a function of carbonated species, concentration of divalent ions, temperature and/or pressure, under mechanical agitation; (b) collecting samples of the aqueous fraction; and (c) performing titration of the collected aqueous phase samples. The present invention further relates to a production water mimicking system comprising: a mechanical agitation system; a resistant material reactor, comprising a single inlet for aqueous phase, organic phase and sample; a gas inlet controller; a heating and temperature control system; and a pressure relief valve sample collection system.

### CROSS-REFERENCES TO RELATED APPLICATIONS

This application claims the benefit of priority to Brazilian Patent Application No. 1020230227848 filed Oct. 31, 2023, the contents of which are hereby incorporated by reference in their entirety for all purposes.

### FIELD OF THE INVENTION

The present invention is part of the field of chemical and oil engineering, more precisely in the field of production water and naphthenic acids, and describes a method for determining the partition of naphthenic acids from a production water mimicking system that considers different variables such as pH, salinity, temperature, alkalinity, pressure and presence of divalent cations. The present invention further relates to a production water mimicking system for evaluating the partition of naphthenic acids.

### BACKGROUNDS OF THE INVENTION

The process of formation of fossil fuels (coal, shale, oil) results in the formation of complex mixtures of hydrocarbons and various organic substances (Barman, B. N. Critical Reviews in Analytical Chemistry. 2000, 30, 75-120). Thus, the composition of these species depends on the historical process of formation of the reserve, the type of oil, as well as the extraction process itself (Barman, B. N. Critical Reviews in Analytical Chemistry. 2000, 30, 75-120; Abdulredha, M. M., Hussain, S. A., Abdullah, L. C. Overview on petroleum emulsions, formation, influence and demulsification treatment techniques. Arabian Journal of Chemistry, 2020, 13, 3403-3428). Understanding the chemical equilibria established in this context is of great importance for the oil and gas industry, given its relevance in the process and its economic impact. However, there is still a need for approaches to quantify chemical compounds in production water.

In general terms, the study of the partitioning phenomenon of acidic species is a topic that has been discussed in recent years (Bertheussen, A.; Simon, S. C.; Sjoblom, J. Equilibrium partitioning of naphthenic acids and bases and their consequences on interfacial properties. Colloids and Surfaces A. 2017, 529, 45-56; Dudek, M., Kancir, E., Øye, G. Influence of the Crude Oil and Water Compositions on the Quality of Synthetic Produced Water. Energy&Fuels, 2017, 31, 3708-3716). The relevance of the topic is directly linked to the volume of production water and the compounds present in this matrix (Zheng, J. et al. Offshore produced water management: A review of current practice and challenges in harsh/Arctic environments. Mar. Pol. Bul. 2016, 104, 7-19), since this system contains a wide range of soluble and insoluble compounds in aqueous media, suspended and dissolved solids, inorganic fractions from sedimentary matrices, in addition to the compounds introduced into the medium during processing.

In the production water matrix, there are important issues that need to be further detailed, such as the influence of salts and pressure during extraction, as well as the mechanisms of the chemical reactions that occur at the interfaces between the aqueous and organic phases. Knowing the characteristics that most influence this phenomenon is essential to be able to direct field work, gaining efficiency and reducing operational costs.

Furthermore, studies along these lines help to understand the interferents that may co-elute in the production water used in the process. In this sense, alternatives to direct disposal into the sea can be considered, such as, for example, filtration or neutralization processes. These evaluations are essential since, according to Duraisamy and collaborators (Duraisamy, R. T., Beni, A. H., Henni, A. State of the art treatment of produced water. In: Elshorbagy, W., Chowdhury, R. K. (Eds.) Water Treatment. InTech, Croatia, Europe, 2013, p. 199-222), more than 77 billion barrels of oil are generated per year from production water. In fact, the range of compounds found in this matrix is high (Petersen, M. A.; Grade, H., 2011. Analysis of steam assisted gravity drainage produced water using two-dimensional gas chromatography with time-of-flight mass spectrometry. Ind. Eng. Chem. Res. 50 (21): 12217-12224), not being restricted to demulsifiers and antiscalants, but also surfactants and secondary compounds, many of ionic nature (Abdulredha, M. M., Hussain, S. A., Abdullah, L. C. Overview on petroleum emulsions, formation, influence and demulsification treatment techniques. Arabian Journal of Chemistry, 2020, 13, 3403-3428). As the partition equilibrium of these species begins to be elucidated (Havre, T. E.; Sjoblom, J.; Vindstad, J. E. Oil/Water-Partitioning and Interfacial Behavior of Naphthenic Acids. Journal of Dispersion Science and Technology. 2003, 24, 789-801; Dudek, M., Kancir, E., Øye, G. Influence of the Crude Oil and Water Compositions on the Quality of Synthetic Produced Water. Energy&Fuels, 2017, 31, 3708-3716), the industry has realized its relevance (Neff, J. M. et al. Oil well produced water discharges to the North Sea, Part II: comparison of deployed mussels (Mytilus edulis) and the DREAM model to predict ecological risk. Mar. Environ. Res. 2006, 62, 224-246; Zheng, J. et al. Offshore produced water management: A review of current practice and challenges in harsh/Arctic environments. Mar. Pol. Bul. 2016, 104, 7-19) and impact, although not yet systematically.

An alternative to the treatment of production water currently used is the use of demulsifiers. According to Chen et al (Chen, W-H.; Chen, C-J.; Hhung, C-I. Taguchi approach for co-gasification optimization of torrefied biomass and coal. Bioresource Technology, 2013, 144, 615-622), it is expected that the removal of the emulsions using chemical demulsifiers is an effective way to treat production water, since it would avoid the formation of emulsions and the problems associated with the treatment and transportation of crude oil (Abdulredha, M. M., Hussain, S. A., Abdullah, L. C. Overview on petroleum emulsions, formation, influence and demulsification treatment techniques. Arabian Journal of Chemistry, 2020, 13, 3403-3428). On the other hand, the chemical equilibria established in the presence of these substances are still neglected and, therefore, it is not possible today to estimate how and to what extent migrations of compounds such as organic acids occur depending on the chemical matrices and the physical-chemical parameters involved with the production water.

The acidic species that occur in production waters tend to partition, that is, establish a partition equilibrium, between the two phases of this system: the aqueous phase (of a hydrophilic nature) and the oily phase (of a hydrophobic nature). On the other hand, recent studies (Taylor, S. E.; Chu, H. T. Metal Ion Interactions with Crude Oil Components: Specificity of Ca2+ Binding to Naphthenic Acid at an Oil/Water Interface. Colloids and Interfaces. 2018, 2, 1-21) highlight the presence of these naphthenic species also at the interface formed between the aforementioned phases, largely due to the chemical structure of the species itself, as well as the results of the conditions and chemical variables of the system (Eke, W. I.; Victor-Oji, C.; Akaranta, O. Oilfield metal naphthenate formation and mitigation measures: a review. Journal of Petroleum Exploration and Production Technology. 2020, 10, 805-819). In short, according to the same authors, the main factors that influence the equilibrium between the phases are the size of the molecule, its chemical structure, the pH of the aqueous fraction, the concentration of salts (mono and divalent species, of organic and inorganic nature), temperature and pressure of the medium.

The expression “naphthenic species”, used as a generalization of the acidic species in this matrix, describes cyclic acids with different derived structures, which represent the majority of oil carboxylic acids. In general, these species have between 10 and 15 carbon atoms, containing between zero and six naphthenic rings, and with the carboxylic acid group attached to the rings and also having some lateral alkyl chain (Eke, W. I.; Victor-Oji, C.; Akaranta, O. Oilfield metal naphthenate formation and mitigation measures: a review. Journal of Petroleum Exploration and Production Technology. 2020, 10, 805-819; Wu, C.; Visscher, A.; Gates. I.D. On naphthenic acids removal from crude oil and oil sands process-affected water. Fuel. 2019, 253, 1229-1246). However, the expression is applied generically in the oil industry, also referring to other types of acids, such as, for example, aliphatic and aromatic acids, as long as they are associated with oil.

In order to evaluate the chemical phenomena involved in the processes through which the production water (and the acidic species present therein) passes, it is necessary to systematically understand the effect of the variables on the equilibrium of the acidic species between the organic and aqueous phases. Once the understanding of the effect of these variables is constructed, it becomes possible to build chemical-mathematical models, preferably with a chemometric bias, that ensure the reliability of the results and offer the capacity to predict the phenomena studied. In short, these models can contribute to the comparison of the influence and interactions between variables, using model substances in simplified systems (e.g., heptane and toluene, in different proportions) (Visscher, J.; Gaakeer, W. A.; Mendoza, P. G.; de Croon, M. H. J. M.; van der Schaaf, J.; Schouten, J. C. Liquid-Liquid Extraction Systems of Benzoic Acid in Water and Heptane, Methylbenzene, or Trichloroethylene as Cosolvent. Journal of Chemical and Engineering Data. 2011, 56, 3630-3636; Choe, J.; Kim, I.; Kim, S. Y.; Song, K. H. Liquid-Liquid Equilibria for the Ternary Systems Water+n-Pentanoic Acid with n-Heptane or Dichloromethane at 298.2K. Journal of Chemical and Engineering Data. 2008, 53, 1199-1202), in order to be able to make adaptations and reliably evaluate real systems (crude oil).

Furthermore, and assuming that the equilibrium of acidic species present in matrices containing oil and production water depends directly on the characteristics of the system, knowledge of the contribution (individual and synergistic) of each chemical variable to this partition may be essential, not only to predict the behavior of a specific system but also to possibly intervene in the system in order to shift the equilibrium towards the presence of these molecules in the organic phase (Eke, W. I.; Victor-Oji, C.; Akaranta, O. Oilfield metal naphthenate formation and mitigation measures: a review. Journal of Petroleum Exploration and Production Technology. 2020, 10, 805-819), which in turn would imply a reduction in the pollutant load in waste from the oil industry, for example.

Some studies (Bertheussen, A.; Simon, S. C.; Sjoblom, J. Equilibrium partitioning of naphthenic acids and bases and their consequences on interfacial properties. Colloids and Surfaces A. 2017, 529, 45-56; Taylor, S. E.; Chu, H. T. Metal Ion Interactions with Crude Oil Components: Specificity of Ca2+ Binding to Naphthenic Acid at an Oil/Water Interface. Colloids and Interfaces. 2018, 2, 1-21; Eke, W. I.; Victor-Oji, C.; Akaranta, O. Oilfield metal naphthenate formation and mitigation measures: a review. Journal of Petroleum Exploration and Production Technology. 2020, 10, 805-819) indicate that the impact of this understanding can go far beyond the prevention of polluting loads in wastes, as it can help in understanding and preventing other operational problems. The presence of naphthenic species can cause corrosion in equipment, and the reaction between these substances present in oil and bicarbonates in formation water can lead to the formation of naphthenates under conditions of high pH, commonly generated by depressurization during the production process (Bertheussen, A.; Simon, S. C.; Sjoblom, J. Equilibrium partitioning of naphthenic acids and bases and their consequences on interfacial properties. Colloids and Surfaces A. 2017, 529, 45-56; Taylor, S. E.; Chu, H. T. Metal Ion Interactions with Crude Oil Components: Specificity of Ca2+ Binding to Naphthenic Acid at an Oil/Water Interface. Colloids and Interfaces. 2018, 2, 1-21). Such naphthenates tend to accumulate in equipment, especially on the surface, more specifically at the water-oil interface, after combining with sodium and/or calcium ions present in the water, as in the case of low-pressure separators, desalters, heat exchangers, and filters (Eke, W. I.; Victor-Oji, C.; Akaranta, O. Oilfield metal naphthenate formation and mitigation measures: a review. Journal of Petroleum Exploration and Production Technology. 2020, 10, 805-819; Wu, C.; Visscher, A.; Gates. I. D. On naphthenic acids removal from crude oil and oil sands process-affected water. Fuel. 2019, 253, 1229-1246).

However, a systematic evaluation of each environmental and process variable is still necessary, since, when evaluated in isolation, the understanding of the system is not complete. For example, the pH of the aqueous phase is also defined by the pressure of carbon dioxide (CO₂) in the system, a factor that becomes more relevant when considering the high levels of CO₂ in pre-salt wells. An additional example is the salinity of the medium, which can contribute to the establishment of partition equilibrium both by increasing the ionic strength of the medium and by the presence of different divalent species that can, through specific interaction with the other species present, contribute differently to the formation of precipitates in the form of naphthenates (Taylor, S. E.; Chu, H. T. Metal Ion Interactions with Crude Oil Components: Specificity of Ca2+ Binding to Naphthenic Acid at an Oil/Water Interface. Colloids and Interfaces. 2018, 2, 1-21).

Additionally, the presence of acidic species raises relevant environmental issues due to the toxicity of certain families in aquatic environments, as well as the stabilization of emulsions with the potential to generate operational problems (Saliu, F.; Pergola, R. D. Organic bases, carbon dioxide and naphthenic acids interactions. Effect on the stability of petroleum crude oil in water emulsions. Journal of Petroleum Science and Engineering. 2017, 163, 177-184; Bakke, T.; Klungsoir, J.; Sanni, S. Environmental impacts of produced water and drilling waste discharges from the Norwegian offshore petroleum industry. Marine Environmental Research. 2013, 92, 154-169). In particular, Brazil has a lower tendency in terms of the presence of these acids in its oil production context due to the pre-salt layer, whose chemical characteristics are dictated by the predominance of smaller, non-aromatic species (making the oil “lighter”). However, even in this case, a more careful investigation of the partition equilibrium of acidic species is necessary, as mentioned above, both from an operational and environmental point of view. This is further aggravated by the high presence of CO₂ in some of the producing fields, which influences the partition equilibrium.

### STATE OF THE ART

Some documents in the state of the art address to the problem of determining the partition of naphthenic acids in produced water, such as:

The doctoral thesis titled “Naphthenic acid solubility in produced water and their interactions with divalent cations” discloses a study conducted on the partition of naphthenic acids in oil and water. The document, based on a literature review, teaches that the partition equilibrium is affected by factors such as pH, pressure, temperature and salinity of the aqueous phase, that the presence of CO₂ alters the pH of the medium, leading to an alteration in the partition, and that divalent cations have a great influence on the partition equilibrium. In the experiments reported in this document, mixtures of aqueous phase and organic phase of heptane with naphthenic acids were prepared under agitation at 200 rpm for 24 hours. The naphthenic acid content was determined by ultraviolet (UV) spectroscopy (BERTHEUSSEN, A. Naphthenic acid solubility in produced water and their interactions with divalent cations”. Norwegian University of Science and Technology. 2018. 217 p.).

However, the studies reported in the aforementioned document do not use a systematic approach to evaluate the partition equilibrium and in accordance with the sequential steps of the present invention. The systematization of the present invention allows obtaining results that are (a) reproducible and representative of field conditions, (b) quantitative and not just qualitative, and (c) that can be used as a parameter for comparison between the chemical compositions and process parameters of different oil reserves in the world.

Furthermore, the content of the aforementioned document conceptually clarifies the phenomena that occur in water/oil systems in the oil and gas segment. With the advent of the present invention, knowledge about such phenomena has been expanded. For example, the method of the invention has the ability not only to predict the “salting out” effect of naphthenic compounds under increasing salinity conditions, but also to quantify the effect based on the chemical structure of the compounds. Likewise, the method allows the quantification of the effect of pressure on the partition, which is described in the document as “of minor relevance”. Furthermore, the method of the present invention demonstrated that such effect is reversible.

Additionally, the method of the present invention allows the evaluation of the effect of pressure associated with other variables such as temperature and the presence of alkalinity in the aqueous phase, something not reported in the literature.

The ability to perform studies that evaluate synergistic effects on the partitioning of naphthenic acids is scarce in the literature. The synergistic effect between variables occurs naturally in production water, and bench studies do not usually consider this phenomenon, opting to simplify the studies by isolating the variables. The present invention provides a method and a system that allow the evaluation of such synergistic effect on the partitioning of naphthenic acids in the production water.

Furthermore, the aforementioned document uses UV spectroscopy to determine the compounds. Such determination is nonspecific, unlike the titrimetry used in the present invention, which determines species characterized by the carboxylic acid group, in a more specific way than UV spectroscopy for the purpose of the invention.

In turn, the document titled “Oil/Water-Partitioning and Interfacial Behavior of Naphthenic Acids” reports a study whose objective was to determine the partition constant of naphthenic acids from crude oil, identifying the pKa of 4.9, in which the concentration of naphthenic acids was determined by chromatography. The document proposes a way to calculate the amount of naphthenic acids in the production water that presented good correlation with the values measured experimentally.

The quantification technique used in this document is different from that of the present invention and consists of gas and liquid chromatography, both with mass spectrometry detection, for the evaluation of naphthenic species with 10 to 16 carbons. The identification of the species is done quantitatively, but through an analytical principle different from titrimetry, which prioritizes the global effect when measuring acid/base neutralization in a previously factored liquid medium.

Furthermore, the main difference between the present invention and the aforementioned document lies in the procedural approach. Although the phenomenon evaluated is the same, the objective of the study reported in the document was different from the present invention, since it intervened on the pH in order to determine its effect on the migration of the target compounds without considering the usual conditions experienced in the field, such as salinity, temperature, pressure and alkalinity.

While the work reported in the document cited in paragraph [021] uses the pH parameter (arbitrarily selected) to influence the partition phenomenon and determine the behavior of the species under these conditions, the method of the present invention standardizes the physical-chemical variables in order to mimic field conditions, and then observe their effect (individual and synergistic) on the compounds of interest.

Furthermore, the present invention employs an inert/oxidative atmosphere, allowing the quantification of the partition equilibrium under conditions not only at the surface (e.g., 0.1 MPa) but also considering that the environment may be pressurized (e.g., up to 1 MPa). The method also allows the use of CO₂, which can act as a chemical equilibrium modification agent as it alters the ratio of radicals (i.e., reactive species) present in the aqueous phase.

The document titled “Simple Method to Determine the Partition Coefficient of Naphthenic Acid in Oil/Water” discloses a study to determine the partition coefficient of naphthenic acids in oil and water mixtures. In this document, the partition in a mixture of water and n-decane was evaluated, under different pH values. To determine the naphthenic acid contents, colorimetric titration methods and those by Karl Fischer were used (BITSCH-LARSEN, A.; ANDERSEN, S. I. Simple Method to Determine the Partition Coefficient of Naphthenic Acid in Oil/Water. J. Chem. Eng. Data 2008, 53(10): 2272-2274).

As in the previous case, the study reported in the document cited in paragraph [026] uses arbitrarily selected pH values to carry out experiments to determine the partition equilibrium. The present invention, on the other hand, standardizes the physical-chemical variables in order to mimic field conditions, that is, under pH conditions that would actually be encountered, in order to then observe their effect (individual and synergistic) on the compounds of interest.

Furthermore, the use of a 120-liter reaction tank without agitation, since emulsified systems are used in the reported experiments, leads to a certain loss of sample representativeness and quantification capacity, which does not occur in the method of the present invention. Although the determination of the partition coefficient of the naphthenic species has been proven, the reduced solubility of n-decane in water, the scale of the operation, and the organic solvent n-decane, without practical representativeness, make the system limited to the sphere of theoretical knowledge.

In turn, the document titled “Protocol for Preparing Synthetic Solutions Mimicking Produced Water from Oil and Gas Operations” reports a process for preparing production water synthetically, that is, a mixture of water and oil that mimics production water and allows evaluating and predicting results for the treatment of production water based on said mimetic mixtures (DARDOR, D.; AL-MASS, M.; MINIER-MATAR, J.; JANSON, A.; SHARMA, R.; HASSAN, M. K.; AL-MAADEED, M. A. A.; ADHAM, S. Protocol for Preparing Synthetic Solutions Mimicking Produced Water from Oil and Gas Operations. ACS OMEGA 2021, 6(10): 6881-6892).

This document reinforces the need in the state of the art to obtain data on production water with greater representation of real field conditions. However, it does not provide any approach to quantifying the partition equilibrium of naphthenic acids, much less about the various parameters considered simultaneously in the present invention.

The document titled “Naphthenic Acids: Formation, Role in Emulsion Stability, and Recent Advances in Mass Spectrometry-Based Analytical Methods” is a review paper about naphthenic acids. In particular, the document addresses the problems caused by naphthenic acids, their ability to act as a surfactant, forming and stabilizing emulsions in oil and water systems, and the analytical methods used for the qualitative analysis of these acids. The document further discloses that naphthenic acids are the main acidic components of oil and the potentiometric titration method is the established standard for determining TAN (FACANALI, R.; PORTO, N. A.; CRUCELLO, J.; CARVALHO. R. M.; VAZ, B. G.; HANTAO, L. W. Naphthenic Acids: Formation, Role in Emulsion Stability, and Recent Advances in Mass Spectrometry-Based Analytical Methods. 2021. 15 p.)

As highlighted in the document mentioned in paragraph [031], largely due to the experimental complexity and high cost inherent in chromatographic techniques, there is a great need to develop alternative analytical methods that allow the qualification and quantification of naphthenic species in oil and production water. Despite the importance of this class of compounds for the operational routine of production water, the demand for understanding the phenomenon remains high, as it requires the proposal of new experimental methodologies, not just analytical ones. In other words, there is a need to mimic the phenomenon in order to better understand the impact that the physical-chemical factors have on the migration of these species between the aqueous and organic phases.

Furthermore, TAN values and the quantity of naphthenic species are correlated, but they are not chemical synonyms. To evaluate naphthenic species, both the identification of each compound (such as chromatography) and joint evaluation (typical of titrimetry) can be used.

The document titled “Naphthenic Acids Aggregation: The Role of Salinity” reports a study of the influence of salinity on the behavior of naphthenic acids (partition coefficient and aggregation), in pure water, low salinity and high salinity environments, through a computational method (CUNHA, D.; FERREIRA, L. J.; ORESTES, E.; COUTINHO-NETO, M. D.; DE ALMEIDA, J. M.; CARVALHO, R. M.; MACIEL, C. D.; CURUTCHET, C.; HOMEM-DE-MELLO, P. Naphthenic Acids Aggregation: The Role of Salinity. Computation 2022, 10(10): 170-184).

In the document cited in the previous paragraph, the complexity of the phenomenon of partition equilibrium of the naphthenic acids is addressed to by means of molecular modeling. However, the molecular modeling approach necessarily requires the experimental validation of the values and predictions of the chemical-mathematical models, under penalty of decorrelating theory and practice.

Thus, the design, validation and use of a control system that mimics relevant process conditions of the present invention is important even for strategies that use computational models.

In turn, patent document WO 2014/036109 A1, titled “INHIBITORS FOR ORGANICS SOLUBILIZED IN PRODUCED WATER”, discloses naphthenic acid inhibitors for the treatment of production water. The document teaches that the partitioning of naphthenic acids into water and oil is influenced by several factors such as pH, pressure and CO₂ (which influences pH and pressure).

The aforementioned document focuses on determining water-soluble organic compounds, defining them as compounds present in the oily fraction and that have sufficient polarity to have an affinity for the aqueous fraction, among them, the carboxylic acids. The objective of the document is to present a way to determine the presence of these species by associating an active surface with functional groups that selectively retain these compounds, so that they can be subsequently quantified by gravimetry.

The invention described in the aforementioned document has a similar objective to that of the present invention, although with a more diffuse focus, because it does not identify naphthenic species with structures with groups such as esters and ketones that are equally (and partially) soluble in aqueous media. In addition, the gravimetric methodology is different from the titrimetric approach due to the analysis principle and, consequently, the sample preparation required. Additionally, gravimetric procedures are based on prior liquid-liquid extraction. It is known from the literature that such procedures lack reproducibility.

Furthermore, patent document WO 2011/032227 A1, titled “METHODS FOR SELECTION OF A NAPHTHENATE SOLIDS INHIBITOR AND TEST KIT, AND METHOD FOR PRECIPITATING NAPHTHENATE SOLIDS”, discloses a method for selecting a naphthenic solids inhibitor for application in liquid hydrocarbons. The document suggests testing different conditions on naphthenic acids for application of a treatment, by evaluating the effect on a mixture of oil and water in which naphthenic acids are present.

The formation of naphthenates from the reaction between divalent cations and naphthenic structures typical of matrices such as production water is reported in the scientific literature. The group called “naphthenic acids” encompasses quite diverse chemical structures, within the classification of carboxylic acids, which limits the production capacity of individual/simple standards or reference standards. The naphthenates described in this document are high molecular weight structures, with 80 carbon atoms. Therefore, they are different from those that end up partitioning in water, related to the object of the present invention.

In the aforementioned document, with the objective of quantifying the formation of naphthenates, the system buffering strategy is used in order to guarantee minimal pH variation and, thus, not compromise the applicability of the method. Again, here, pH is considered as a predictive physical-chemical variable (i.e., one that influences the partition equilibrium), and not as a parameter to be arbitrarily defined.

Thus, there is a need in the state of the art for the provision of a method that allows the quantitative determination of the partition equilibrium of naphthenic acids in production water, considering the different variables that influence this phenomenon under field conditions.

### SUMMARY OF THE INVENTION

The present invention aims at proposing, firstly, a method for determining the partition of naphthenic acids in a system that mimics production water, comprising the steps of:
a) mixing, in a mimicking system, an aqueous phase and an oily phase comprising acidic species, varying parameters of pH, salinity, alkalinity as a function of carbonated species, concentration of complex-forming divalent ions, temperature and/or pressure, under mechanical agitation;
b) collecting samples, under agitation and/or in the static period, of the aqueous fraction; and
c) performing titration, preferably potentiometric, of the collected aqueous phase samples.

In a second embodiment, the present invention relates to a production water mimicking system (6) comprising: a mechanical agitation system (1); a reactor made of resistant material (2), comprising a single inlet for the aqueous phase, organic phase and sample (21); a gas inlet controller (3), for pressure variation; a heating and temperature control system (4); and a pressure relief valve sample collection system (5).

### BRIEF DESCRIPTION OF THE FIGURES

In order to obtain a full and complete view of the objective of this invention, the Figures to which reference is made are presented, as follows.

FIG. 1 is a graphical representation of the production water mimicking system (6) of the present invention comprising: a mechanical agitation system (1); a reactor made of resistant material (2), comprising a single inlet for the aqueous phase, organic phase and sample (21); a gas inlet controller (3), for pressure variation; a heating and temperature control system (4); and a pressure relief valve sample collection system (5).

FIG. 2 graphically illustrates the percentage recovery of benzoic acid as a function of the time of addition of HCl in samples containing bicarbonate or bicarbonate and carbonate in saline solution (3.5% NaCl), wherein B represents the medium with bicarbonate and B+C represents the medium with bicarbonate and carbonate.

FIG. 3 graphically illustrates the percentage recovery of pentanoic acid as a function of the time of addition of HCl in samples containing bicarbonate or bicarbonate and carbonate in saline solution (3.5% NaCl), wherein B represents the medium with bicarbonate and B+C represents the medium with bicarbonate and carbonate.

FIG. 4 refers to the Pareto diagram regarding the effect of pressure and temperature on the partition equilibrium of benzoic acid.

FIG. 5 refers to the graphical representation of the curve of the effect of pressure and temperature on the partition equilibrium of benzoic acid.

FIG. 6 refers to the response surface of the evaluation model of the effect of pressure and temperature on the partition equilibrium of benzoic acid.

FIG. 7 refers to the Pareto diagram regarding the effect of pressure and temperature on the partition equilibrium of pentanoic acid.

FIG. 8 refers to the graphical representation of the curve of the effect of pressure and temperature on the partition equilibrium of pentanoic acid.

FIG. 9 refers to the Pareto diagram regarding the effect of pressure and temperature on the partition equilibrium of cyclohexanepentanoic acid.

FIG. 10 refers to the graphical representation of the curve of the effect of pressure and temperature on the partition equilibrium of cyclohexanepentanoic acid.

FIG. 11 refers to the response surface of the model for evaluating the effect of pressure and temperature on the partition equilibrium of cyclohexanepentanoic acid.

FIG. 12 graphically represents the titration profile of the aqueous phase from systems containing acidic species added to the heptol phase, under increasing salinity conditions (3.5-30%, m·v⁻¹) of the aqueous phase.

FIG. 13 graphically represents the results of neutralization titration for comparison of the equilibrium in the increasing presence of calcium ions in the medium containing benzoic acid, pentanoic acid, cyclohexanepentanoic acid, dicyclohexylacetic acid and 5-phenylpentanoic acid.

FIG. 14 graphically represents the neutralization titration results for comparison of equilibrium in the increasing presence of strontium ions in the medium containing benzoic acid, pentanoic acid, cyclohexanepentanoic acid, dicyclohexylacetic acid and 5-phenylpentanoic acid.

FIG. 15 graphically represents the results of neutralization titration in the presence of demulsifiers in the medium containing benzoic acid, pentanoic acid, cyclohexanepentanoic acid, 5-phenylpentanoic acid, in which the dashed line refers to the data with the emulsifier D974 and the dotted line refers to the data with the emulsifier D961.

FIG. 16 graphically represents the results of the neutralization titrations of the study with the addition of tetrabutylammonium chloride to the system containing acidic species.

FIG. 17 graphically represents the results of the neutralization titrations of the study with the addition of trimethylammonium chloride to the system containing acidic species.

FIG. 18 graphically represents the results of the neutralization titrations of the study with the addition of cetyltrimethylammonium chloride to the system containing acidic species.

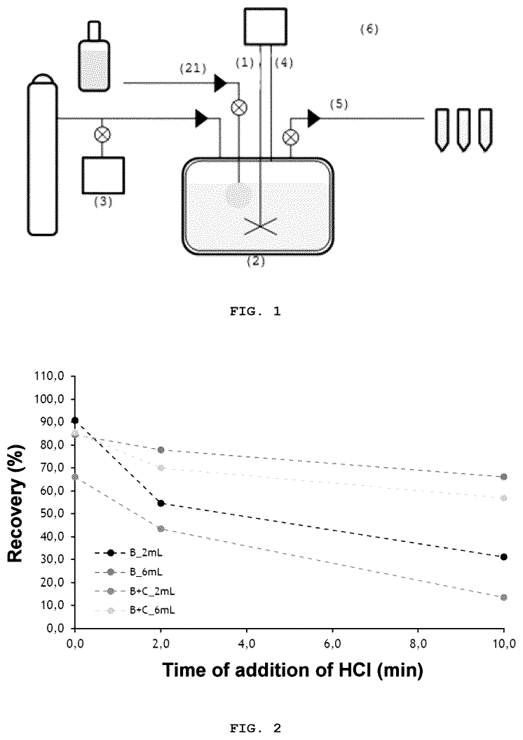

### DETAILED DESCRIPTION OF THE INVENTION

The present invention aims at proposing, firstly, a method for determining the partition of naphthenic acids in a system that mimics production water, comprising the steps of:
a) mixing, in a mimicking system, an aqueous phase and an oily phase comprising acidic species, varying parameters of pH, salinity, alkalinity as a function of carbonated species, concentration of complex-forming divalent ions, temperature and/or pressure, under mechanical agitation;
b) collecting samples, under agitation and/or in the static period, of the aqueous fraction; and
c) performing titration, preferably potentiometric, of the collected aqueous phase samples.

In a preferred embodiment, step a) is performed under mechanical agitation with rotations between 200 and 1000 rpm, controlled by analog or digital controllers. Preferably, the mixture is kept under agitation for 2 to 30 minutes, even more preferably from 2 to 10 minutes, depending on the oily matrix being evaluated.

In a preferred embodiment, step a) is carried out in reactors with volumes between 100 and 600 mL. Preferably, the reactors are made of material resistant to corrosion by oxidative and/or ionic species. Even more preferably, the reactor is made of steel alloys or glass.

In a preferred embodiment, step a) is carried out under a pressure ranging from 0.1 MPa to 1 MPa. Preferably, the pressure alteration is controlled by introducing N₂ or CO₂.

In a preferred embodiment, step a) is carried out at a temperature equal to or up to 60° C. above room temperature. Preferably, step a) is carried out at a temperature of 25° C. to 80° C. Preferably, the temperature is controlled by an analog or digital controller, with a thermostated bath, with a variation of up to 0.5° C.

In an even more preferred embodiment, the ratio between the oily phase and the aqueous phase of the mixture of step a) is 3:1.

In a preferred embodiment, the oily phase comprises heptol. In an alternative preferred embodiment, the oily phase comprises crude oil. Preferably, the heptol comprises a mixture of heptane and toluene in the range of 90% (m/m) heptane to 10% (m/m) toluene up to 10% (m/m) heptane to 90% (m/m) toluene. More preferably, the heptol comprises heptane and toluene in the proportion of 70% (m/m) heptane and 30% (m/m) toluene.

In a preferred embodiment, naphthenic acids are added to the oily phase. Preferably, the added naphthenic acids are benzoic acid, pentanoic acid, cyclohexanepentanoic acid, 5-phenylpentanoic acid and dicyclohexylacetic acid. However, the method and the system disclosed herein are not particularly limited to such acids and are applicable to other naphthenic acids, such as, for example: hexanoic acid, naphthoic acid, cyclohexanoic acid, cyclopentanoic acid, 3-methyl-cyclohexanoic acid, 4-heptylbenzoic acid, 5-phenylpentanoic acid, 3-(3-ethylcyclopentyl)propanoic acid, cyclohexaneethanoic acid, cyclohexanepropanoic acid, cyclohexanebutanoic acid, cyclohexanehexanoic acid, cyclopentylethanoic acid, cyclopentylpropanoic acid, undecanoic acid, dicyclohexylethanoic acid, 4-heptylcyclohexanoic acid, benzylpropanoic acid, benzylpentanoic acid and decahydronaphthoic acid.

In a preferred embodiment, the aqueous phase comprises ultrapure or distilled water.

In an alternative preferred embodiment, step a) allows evaluation of the effect of surfactants, demulsifiers, antiscalants, drilling fluids, among others, from the addition of these components to the system. The addition can be made in either the aqueous or oily phase, depending on the interest in mimicking the system.

In a preferred embodiment, between 30 and 100 mL of the aqueous fraction are collected in step b).

The samples obtained in step b) can either be immediately subjected to analysis in step c) or can be kept refrigerated (4° C. to 8° C.) for later analysis.

Optionally, step b) further includes obtaining samples of the oily phase.

In a preferred embodiment, titration step c) is potentiometric and is performed with a NaOH solution, preferably 0.01 M. In a preferred embodiment, potentiometric titration step c) is performed in an automatic titrator.

In an alternative embodiment, titration step c) is performed by classical titration, using indicators or a pH meter.

In a preferred embodiment, an HCl solution is added to the samples before performing step c) to allow quantification of the acidic species.

In order to confirm that the preparation of the alkaline systems remains within best practices, initial and final pH monitoring is preferably maintained. As a quality control parameter, the minimum acceptable recovery of the acidic species, regardless of the experiment performed in step a), is defined as 90% (m/m), and must be periodically verified.

In a preferred embodiment, the method further comprises a sample preparation step. For oily samples, the sample preparation step comprises mechanical homogenization for 5 to 15 minutes (between approximately 300 rpm and 500 rpm).

The sample preparation step additionally comprises the preparation of the heptol solution, comprising 90% to 10% (m/m) of heptane and 10% to 90% (m/m) of toluene, used as the oily phase.

The sample preparation step further comprises the preparation of the following solutions: (a) titrant solution comprising 0.01 M NaOH; (b) 0.01 M HCl solution; (c) 3.45×10⁻⁵ mol sodium bicarbonate solution; and (d) 3.48×10⁻⁵ mol sodium carbonate and bicarbonate solution.

In an even more preferred embodiment, the titrant solution is prepared by heating the aqueous solution prior to the addition of NaOH, keeping the medium between 90° C. and 85° C. for 30 to 60 min, in order to ensure the decarbonation of the aqueous medium. Subsequently, the solutions must be stored under refrigeration (between 4° C. and 8° C.) in amber bottles.

The HCl solution is used to acidify the system when the control system contains compounds that increase the alkalinity of the medium (for example, sodium carbonate and/or bicarbonate).

In a second embodiment, the present invention relates to a production water mimicking system (6) comprising: a mechanical agitation system (1); a reactor made of resistant material (2), comprising a single inlet for the aqueous phase, organic phase and sample (21); a gas inlet controller (3), for pressure variation; a heating and temperature control system (4); and a pressure relief valve sample collection system (5).

In a preferred embodiment, the production water mimicking system (6) is particularly adapted for the method for determining the partition of naphthenic acids described herein.

In a preferred embodiment, the gas inlet controller (3) is a manual controller by means of a ball valve for piping, preferably made of ¼ stainless steel. The valve may be ⅜″ (0.9525 cm) or ½″ (1.27 cm), without requiring changes in the operating parameters. In an alternative embodiment, the gas inlet controller (3) is automated, with the valves being replaced by units of the same size, responsive to an external controller.

In a preferred embodiment, the mechanical agitation system (1) and the heating and temperature control system (4) are controlled by the same controller, which comprises a bivolt digital reaction controller or similar.

In a preferred embodiment, the reactor made of resistant material (2) has volumes between 100 and 600 mL. Preferably, the reactor (2) is made of material resistant to corrosion by oxidative and/or ionic species. Even more preferably, the reactor (2) is made of steel alloys or glass.

In a preferred embodiment, the heating and temperature control system (4) is an analog or digital controller, with a thermostatic bath, with a maximum variation of up to 2° C.

In a preferred embodiment, the present invention provides a method for measuring the effect of introducing seawater, in an operational environment, on the presence of acidic species in the production water.

The production water mimicking system (6) allows operation with salinities, in the aqueous fraction, between 3.5 and 30% (m·v⁻¹), which simulates the quantity of ions present in seawater (minimum value) up to hypersaline conditions (maximum value) in which salts accumulate due to the well operation. In these cases, naphthenic species can migrate differently between the oily and aqueous phases, generating greater or lesser operational complications.

The production water mimicking system (6), operating at constant temperature (±1.0° C.) and pressure (±0.01 MPa), can be filled with a 300 mL aqueous phase containing an initial salinity of 3.5% (m·v⁻¹), in the presence of naphthenic acid species with a carbon chain of 5 to 12 carbon atoms, in which the oily phase is oil or heptol. The environment may or may not be kept inert by controlling the desired gas inlet, especially to mimic environments with little oxygen present. Thus, for example, it operates for 10 min at 250 rpm, allowing the equilibrium of partition of the acidic species to be reached in order to generate a representative aliquot of the process. Subsequently, an additional experiment with salinity of the aqueous phase at 30% (m·v⁻¹) allows the variation in the behavior of the naphthenic species to be identified. The observed effect is directional and depends on the chemical structure of the compounds present in the production water, with a predominance of the “salting out” effect of up to 400% compared to typical salinity conditions of a marine environment, especially for acidic species with a shorter carbon chain.

Using this type of approach, it is possible to evaluate the influence of the injection water (secondary recovery method) and its interaction with the water/oil present in the reservoir. With this, the partition effect in the production water can be predicted depending on the arrival of the injection water.

In an alternative preferred embodiment, the present invention provides a method for measuring the effect of the presence of calcium on the presence of acidity in production water.

The control system allows operation with the presence of specific ions, such as calcium salts, which are potential naphthenate formers when naphthenic acid species are present. Such naphthenates tend to accumulate, after combining with sodium and/or calcium ions present in the water, in the equipment, especially on the surface, more specifically at the water-oil interface, as in the case of low-pressure separators, desalters, heat exchangers and filters.

To this end, the preparation of the aqueous phase of constant salinity (up to 30%, m·v⁻¹) receives the addition of calcium chloride, and can also receive experiments with additional salts of sodium, strontium and barium, for example. Once the reactor is filled with the aqueous fraction and organic phase, the system operates, for example, for 5 min at 500 rpm, allowing the equilibrium of the partition of the acidic species to be reached in order to generate a representative aliquot of the process.

This experiment allows the evaluation of effects lower concentrations, typically between 200 and 400 mg/L of Ca²⁺, as well as extreme conditions of 9000 mg/L Ca²⁺ in production water, maintaining reliability (throughout the analysis range) on the partition equilibrium established in the control system and which is sampled at the end of the period. The observed effect is directional and depends on the chemical structure of the compounds present in the production water, especially regarding the size of the carbon chain and the existence of charge divalence, with a predominance of increased migration to the aqueous phase of up to 100% for acidic species with a longer carbon chain, and negative (up to 50%) for species with a shorter carbon chain, such as species with up to 6 carbons.

From knowledge of the composition of the water, more specifically the calcium content, it is possible to predict the partition of a given acid into the aqueous phase.

In an alternative preferred embodiment, the present invention provides a method for measuring the effect of the presence of alkalinity sources on the acidity of production water.

The control system allows operation with up to 600 mL of reaction medium, maintaining a 3:1 ratio of aqueous to oily phase. Once agitation is maintained at 500 rpm for 8 min, followed by rest for 10 min, there can be collected aliquots representative of the partition equilibrium of naphthenic acid species in an aqueous environment with the presence of alkalinity sources, represented, for example, by compounds such as sodium carbonate and bicarbonate. Sampling and analysis, respectively, steps b) and c), must be carried out following the operation of the control system, within a maximum time of 60 minutes, in order to ensure that the reaction medium is not altered by the change in the equilibrium of species in the aqueous fraction.

For this purpose, the system is maintained under a constant atmosphere by introducing N₂ or CO₂ at ambient pressures or up to 1 MPa, at a constant temperature set between 25° C. and 80° C. The choice of atmosphere includes controlling the entry of gas into the control system and is related to the environment to be mimicked, i.e., with greater or lesser oxidative power and/or presence of CO₂. Furthermore, up to approximately 3.5×10⁻⁵ mol, preferably 3.48×10⁻⁵ mol, of NaHCO₃ and Na₂CO₃, both responsible for the alkalinity of the reaction medium, are used by preparing aqueous solutions containing one or both NaHCO₃ and Na₂CO₃. In addition to determining the influence of pressure and temperature on partitioning, as well as eventual synergistic effects with alkaline sources added to the aqueous phase, this system allows to understand the effect of the carbon structure of acidic species on their migration tendency, since the process conditions allow the independent migration of compounds depending on their affinity for the aqueous and oily phases.

Environments saturated with CO₂ or salts that generate alkalinity in production water are usual operating conditions. The production water mimicking system (6) indicates that alkalinity increases the migration tendency of acidic species by 100%, especially those with more than 8 carbons. The migration was found to be reversible in experiments with up to 1 MPa, which equally directs the acidic species to the aqueous phase, since once the production water mimicking system is depressurized, the tendency is reversed. The same does not occur (i.e., reversibility) in media with residual alkalinity, making this parameter even more relevant for the dynamics of production water management.

To demonstrate its potential, the present invention will be described in more detail in terms of embodied examples. It should be highlighted that the following description is only intended to clarify the understanding of the proposed invention and to disclose, in more detail, the embodiment of the invention without limiting it to the same. In this way, variables similar to the examples are also within the scope of the invention.

### Example 1: Implementation of Back Titration Methodologies

The neutralization titrations were performed using standardized solutions of 0.01 M HCl and 0.01 M NaOH (the latter, with care for the preparation and standardization of the solution, wherein the ultrapure water used in the preparation of the solution was previously boiled to eliminate CO₂). For the back titration procedure, a series of solution preparations and additional control tests were adopted:
preparation of a ±0.01 M HCl solution (for addition of 2.0-6.0 mL to each 10.0 mL titrated sample) in order to acidify the system after alkalinization with carbonate and/or bicarbonate, in which 2.0 mL were used for the sample containing only the acid, 4.0 mL in the presence of bicarbonate and 6.0 mL in the presence of carbonate and bicarbonate, aiming at maintaining the initial pH between 3 and 4;
triplicate titrations, with ±0.01 M NaOH;
preparation of sodium bicarbonate solutions (3.45×10⁻⁵ mol added to the reactor or used for control tests), followed by acidification and neutralization titration (in triplicate);
preparation of sodium carbonate and bicarbonate solutions (3.48×10⁻⁵ mol added to the reactor or used for control tests);
preparation of a solution with the acidic species (individually), in concentrations of 0.03 M for benzoic and pentanoic acids, and 0.01 M for cyclohexanepentanoic, 5-phenylpentanoic and dicyclohexylacetic acids, all added in the organic phase (heptol 70:30) used in the reactor step, following the previous procedures.

Additionally, and in order to confirm that the preparation of the systems with alkalinity remains within best practices, monitoring of the initial and final pH was maintained in all cases. Control tests aimed at attesting the reliability of the back titration in terms of recovery (%, mol) of the acidic species added in 10.0 mL of water containing the alkalinity sources mentioned above. In these cases, the minimum acceptable recovery was defined as 90%, and was achieved for all acidic species evaluated prior to the study itself.

Standardization of the NaOH and HCl solutions is essential to ensure the reliability of the potentiometric titration procedure, not only to allow accurate calculation of the concentrations of acidic species in the aqueous phase (usually on the mmol scale), but also to ensure that it is possible to measure the effect of process variables. Thus, and considering the standardization procedure with C₈H₅KO₄, Table 1 presents an example of a calculation, illustrating usual values obtained by the described procedure.

In general, if the standard deviation value is considered as a measure of the variability of the procedure between replicates, low dispersion around the mean is noted (values of only 0.15%). In all cases, the variation in the volume of NaOH used between the titration replicates is in the order of 0.2 to 0.4 mL.

TABLE 1
Results of standardization titrations of 0.01M NaOH

| | Mass of | Volume of | NaOH |
|---|---|---|---|
| | potassium | NaOH consumed | concentration |
| Replicates | biphthalate (g) | (mL) | (mol/L) |
| 1 | 0.022 | 13.0 | 0.0082 |
| 2 | 0.021 | 12.8 | 0.0082 |
| 3 | 0.022 | 13.2 | 0.0082 |
| 4 | 0.022 | 13.3 | 0.0082 |
| Mean | 0.022 | 13.1 | 0.0082 |
| Standard | | | 0.15% |
| Deviation | | | |

Thus, the internalization of titrimetric methodologies allows the quantitative determination of the proportion of acids contained in the aqueous phase and, from this measurement, the estimation of the partition equilibrium of acidic species and the effects of each variable on this equilibrium. To this end, the pH delta between the additions of titrant volume is considered, which enables the detection of the equivalence point.

Furthermore, there is a need for acidification of the aliquot (excess acidic solution, secondary standard), known as back titration, and subsequent neutralization with NaOH±0.01 M (secondary standard) for samples containing acids such as cyclohexanepentanoic, 5-phenylpentanoic and dicyclohexylacetic. For these, the equivalence point, in direct titration, approaches the minimum quantification limit of the equipment and the method, precisely due to the low percentage of migration and low solubility in water of the three compounds.

The demand for sample acidification also extends to titrations in alkaline medium. This procedure demonstrated the characteristic profile of the titration in the presence of bicarbonate and carbonate, that is, containing two inflection points relative to the turning points of each phase of the neutralization. It is worth highlighting that the second turning point serves as a parameter for comparison with the system subsequently containing acidic species, since it is the difference in the volume of this point between the experiments that leads to the quantification of the migration of the acidic species added to the organic phase. The extent of this effect and its measurement are thus performed with a minimum reading error of 0.1 mL and rely on experimental replicates (triplicate), in order to evaluate the reliability of the result.

To ensure the reproducibility of the measurements (i.e., standard deviation values below 10%) and to ensure adequate recoveries of the standards (parameter set at 90%), tests were performed to evaluate the influence of the addition time of the acidic solution (HCl±0.01 M) in the aliquot (i.e., titrated solution). FIG. 2 shows the recovery of the acid as a function of the addition time of the HCl solution, added in a medium with bicarbonate or bicarbonate and carbonate. As a rule, the acid (previously standardized HCl solution) is partially consumed by the alkalinity existing in the aqueous medium and its excess is then titrated by the base (previously standardized NaOH).

From FIG. 4, it is possible to note that the recovery decreases as the time in which the acidic solution is added increases. This occurs for both samples, that is, for those containing only bicarbonate (3.45×10⁻⁵ mol) and also for the media with bicarbonate and carbonate (3.48×10⁻⁵ mol). As a possible explanation for this observation, it is understood that the formation of carbonic acid (an unstable species that decomposes producing CO₂) contributes to this pattern of results. The data presented in Table 2 show that at time zero, that is, when the HCl solution is immediately added to the aliquot, there is a higher percentage of acid recovery. The behavior is verified in both alkaline media, and also considering the addition of 2.0 and 6.0 mL of acidic solution. In all cases, the recovery of benzoic acid is lower with 10 min of HCl solution addition time, especially when adding 2.0 mL of this solution.

TABLE 2
Results of experiments with benzoic acid that vary the addition
time and volume of the 0.01M HCl solution to the titrated product

| | Recovery (%) | | | |
|---|---|---|---|---|
| Time | Bicarbonate | | Carbonate + Bicarbonate | |
| (min) | 2 mL (HCl) | 6 mL (HCl) | 2 mL (HCl) | 6 mL (HCl) |
| 0 | 90.7 | 84.4 | 66.0 | 85.4 |
| 2 | 54.5 | 77.8 | 43.4 | 70.1 |
| 10 | 31.1 | 66.0 | 13.5 | 57.0 |

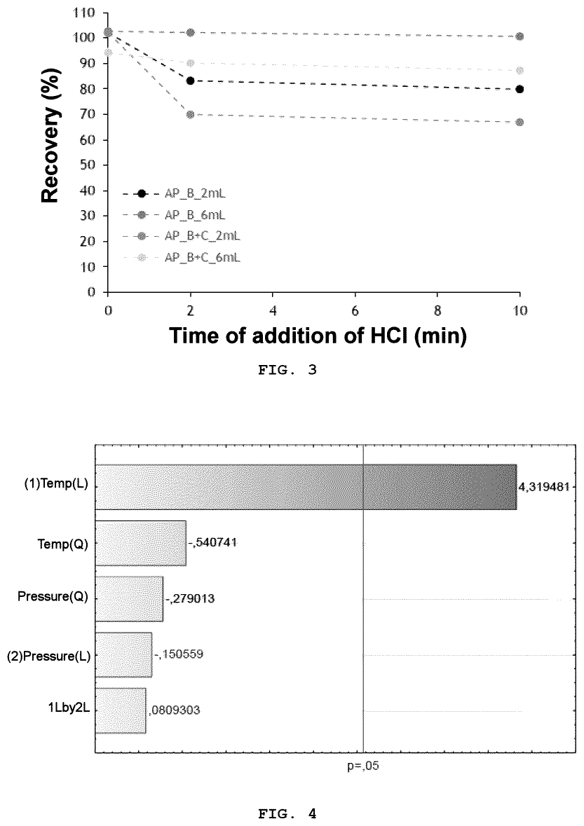

Similarly, FIG. 3 presents the percentage recovery of pentanoic acid as a function of the addition time of the HCl solution. It can be noted that there is a decrease in recovery when slowly adding 2.0 mL of the acidic solution, while there is no observable alteration to the recovery of the acidic species when adding 6.0 mL of HCl. However, as before, the samples at time zero obtained a higher percentage of recovery, that is, between 94.5 and 100.0% (in moles).

Table 3 shows the results for the addition of acid to samples containing alkalinity in the presence of pentanoic acid, at different times. Here, the availability of H+ ions was greater than in the previous experiment, thus resulting in greater recovery even with 10-min intervals for the addition of the HCl solution to the system. Therefore, although to a lesser extent than the pattern observed for benzoic acid, this result corroborates the understanding that the addition of HCl should be done immediately, in all cases.

TABLE 3
Results of experiments with pentanoic acid that vary addition
time and volume of 0.01M HCl solution to the titrated product

| | Recovery (%) | | | |
|---|---|---|---|---|
| Time | Bicarbonate | | Carbonate + Bicarbonate | |
| (min) | 2 mL (HCl) | 6 mL (HCl) | 2 mL (HCl) | 6 mL (HCl) |
| 0 | 100.0 | 100.0 | 100.0 | 94.5 |
| 2 | 83.3 | 100.0 | 70.0 | 90.4 |
| 10 | 79.9 | 100.0 | 66.9 | 87.5 |

As can be seen in Tables 2 and 3, all recovery values, with the addition of 2.0 mL of HCl, are greater than 90%, meeting the necessary requirement to demonstrate the robustness of the back titration and the reliability of the measurement. Optimizing the amount of H+ ions added to the medium is relevant to ensure that there is no excess of this species to the point of “masking” the presence of the acidic species of interest, while at the same time maintaining it above the minimum identifiable by the technique. The immediate addition of 2.0 mL of acidic solution was then adopted consistently in the other examples described here.

### Example 2: Partition Equilibrium Under the Influence of Pressure and Temperature

Three multivariate experimental designs (central composite designs) were performed in which the variables pressure (up to 1 MPa) and temperature (between 25° C. and 80° C.) were considered independent variables, with the migration of three acidic species as the response factor: benzoic, pentanoic and cyclohexanepentanoic acid. For this purpose, 5 levels were defined and gave rise to 10 experiments for each acidic species (Table 4), added to two control experiments (with higher pressure and temperature, as well as in the absence of pressure and at room temperature).

TABLE 4
Experimental design for evaluating the effect of pressure and
temperature on the partition equilibrium of acidic species

Levels

| Variable | −2 | −1 | 0 | 1 | 2 |
|---|---|---|---|---|---|
| Temperature (° C.) | 25.00 | 38.00 | 52.00 | 66.00 | 80.00 |
| Pressure (MPa) | 0.00 | 0.25 | 0.50 | 0.75 | 1.00 |

The experiments were performed with constant salinity of 3.5% (m·v⁻¹), constant heptol fraction (70:30, for heptane and toluene), and acidic species concentration of 0.03 M for benzoic and pentanoic acid, and 0.01 M for cyclohexanepentanoic acid. The results were evaluated with 95% confidence, using a response surface to estimate the maximum observable effect in each case, provided that the mathematical correlation was equal to or greater than R²: 0.75.

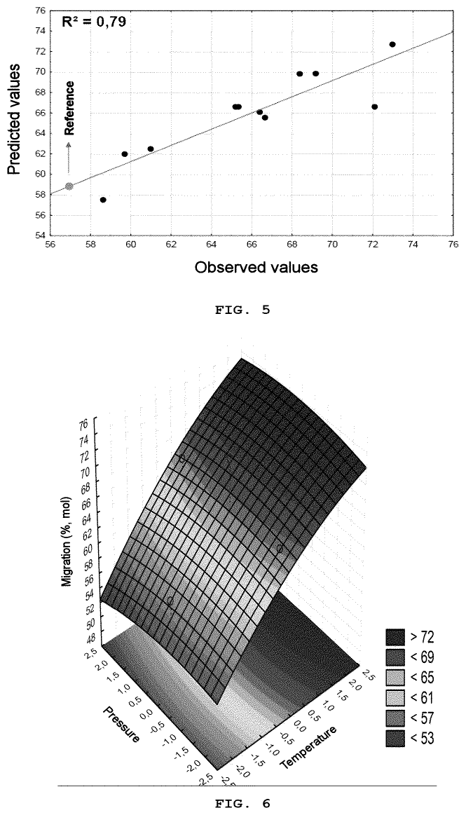

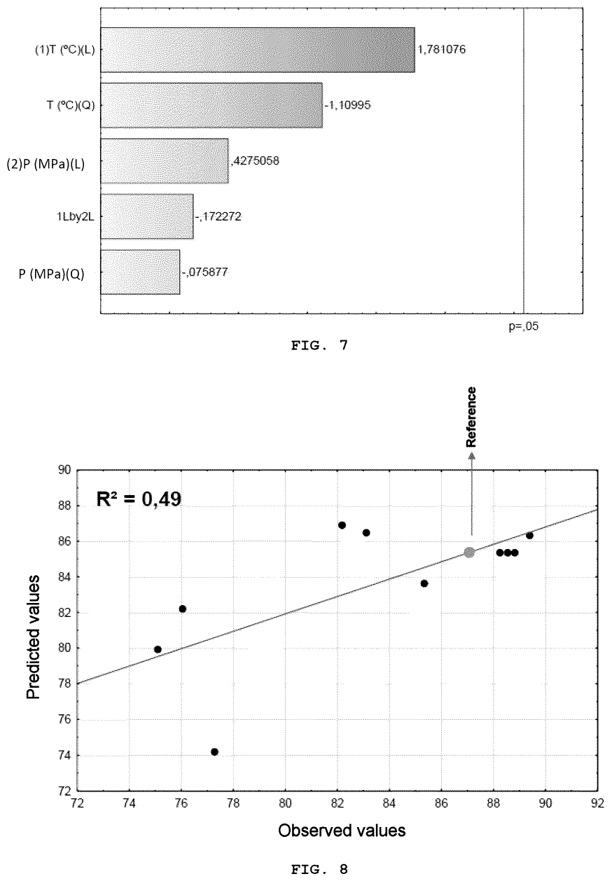

Table 5 presents the results for the experiments with benzoic acid, which show that the variation in migration to the aqueous phase always had an increasing effect, ranging from 4.6 to 27.9% above the results in control systems. In this case, the maximum migration (in %, mol) did not reach 100% in any case, although it had the opposite direction to the other parameters, such as salinity (Example 4) and the presence of calcium and strontium ions (Example 5). Considering that the data modeling generated a correlation of R²: 0.79 (FIG. 5), with normal distribution of the residues (data not shown), the most significant effect can be evaluated, here being temperature (FIG. 4). In this way, the increase in temperature generated a direct effect (i.e., linear) on the increase in the migration of benzoic acid to the aqueous phase.

TABLE 5
Results of the experimental design for evaluating the effect of pressure
and temperature on the partition equilibrium of benzoic acid

| | | | Migration (%, mol) | | |
|---|---|---|---|---|---|
| | | | | | Increment |
| | Temperature | Pressure | | | (related to |
| | (° C.) | (MPa) | Measured | Predicted | control) |
| 1 | 38 | 0.25 | 61.0 | 62.5 | 6.9 |
| 2 | 66 | 0.25 | 69.2 | 69.8 | 21.2 |
| 3 | 38 | 0.75 | 59.7 | 62 | 4.6 |
| 4 | 66 | 0.75 | 68.4 | 69.8 | 19.8 |
| 5 | 25 | 0.5 | 58.6 | 57.5 | 2.7 |
| 6 | 80 | 0.5 | 73.0 | 72.7 | 27.9 |
| 7 | 52 | 0.1 | 66.4 | 66.1 | 16.4 |
| 8 | 52 | 1 | 66.7 | 65.5 | 16.8 |
| 9 | 52 | 0.5 | 65.3 | 66.6 | 14.5 |
| 10 | 52 | 0.5 | 65.2 | 66.6 | 14.3 |
| 11 | 80 | 1 | 72.1 | 66.6 | 26.3 |
| 12 | 20 | 0.1 | 57.0 | — | — |

The response surface (FIG. 6) for this model indicates that temperature can increase the migration of the acidic species, but this effect is limited and independent of the pressure exerted on the medium. This finding should be considered, therefore, in view of the perspective that with the decrease in temperature when removing the system from the well (for example), this effect is negligible (i.e., reversible).

The same experimental approach performed in the presence of pentanoic acid (Table 6), on the other hand, resulted in experiments with a small increase (up to 3.1% mol) and, above all, a decrease in the tendency for the acidic species to migrate (up to 13.3% mol). This behavior, considering its extent, is at lower levels than the other physical-chemical parameters evaluated in the examples presented herein (for pentanoic acid). These differences, considering that the model system has its use standardized, as well as the titration methodology (direct or indirect), indicate that the use of target compounds with different chemical structures (for example, chain size and structural conformation) is relevant to generate greater understanding of the chemical system of interest.

TABLE 6
Results of the experimental design for evaluating the effect of pressure
and temperature on the partition equilibrium of pentanoic acid

| | | | Migration (%, mol) | | |
|---|---|---|---|---|---|
| | | | | | Increment |
| | Temperature | Pressure | | | (related to |
| | (° C.) | (MPa) | Measured | Predicted | control) |
| 1 | 38 | 0.25 | 75.1 | 79.9 | −13.3 |
| 2 | 66 | 0.25 | 83.1 | 86.5 | −4.1 |
| 3 | 38 | 0.75 | 76.1 | 82.2 | −12.2 |
| 4 | 66 | 0.75 | 82.2 | 86.9 | −5.2 |
| 5 | 25 | 0.5 | 77.3 | 74.2 | −10.8 |
| 6 | 80 | 0.5 | 87.1 | 85.5 | 0.5 |
| 7 | 52 | 0.1 | 85.3 | 83.6 | −1.5 |
| 8 | 52 | 1 | 89.4 | 86.3 | 3.1 |
| 9 | 52 | 0.5 | 88.5 | 85.4 | 2.2 |
| 10 | 52 | 0.5 | 88.2 | 85.4 | 1.8 |
| 11 | 80 | 1 | 88.8 | 85.4 | 2.5 |
| 12 | 20 | 0.1 | 86.6 | — | — |

Based on the results above, the data modeling generated a low correlation between the measured and predicted values, R²: 0.49 (FIG. 8), with no significant factor to explain the results. In this way, despite the clear differences in relation to the profile observed for benzoic acid, it is not possible to state that, statistically, there is a considerable effect on the migration of this acid under the effect of pressure and temperature (within the evaluated range). The experiments were repeated with higher residue values, but this did not increase the correlation value beyond that already indicated, which may occur due to the synergism between the variables modeled herein and other parameters that may be ignored (whether physical or chemical).

Still using the multivariate approach, experiments focusing on cyclohexanepentanoic acid were performed and resulted in a significant and more extensive effect in relation to the two previous acidic species. Here, the variation in migration to the aqueous phase was −42.2% (in moles) to an increase of 157.4% (in moles), that is, it had both directions depending on the experimental conditions (Table 7). This variation, moreover, indicates that the acidic species is more susceptible to the effect of both parameters, and herein the modeling of the data results in a gain in understanding the partition equilibrium in acidic species with a larger carbon chain size.

TABLE 7
Results of the experimental design for evaluating
the effect of pressure and temperature on the partition
equilibrium of cyclohexanepentanoic acid

| | | | Migration (%, mol) | | |
|---|---|---|---|---|---|
| | | | | | Increment |
| | Temperature | Pressure | | | (related to |
| | (° C.) | (MPa) | Measured | Predicted | control) |
| 1 | 38 | 0.25 | 2.3 | 1.7 | −42.22 |
| 2 | 66 | 0.25 | 2.5 | 4.4 | −37.25 |
| 3 | 38 | 0.75 | 8.2 | 5.1 | 104.3 |
| 4 | 66 | 0.75 | 8.3 | 7.7 | 106.79 |
| 5 | 25 | 0.5 | 2.4 | 4.0 | −39.73 |
| 6 | 80 | 0.5 | 10.3 | 9.3 | 157.42 |
| 7 | 52 | 0.1 | 2.8 | 1.9 | −29.8 |
| 8 | 52 | 1 | 7.1 | 8.6 | 76.99 |
| 9 | 52 | 0.5 | 2.6 | 3.5 | −34.77 |
| 10 | 52 | 0.5 | 3.1 | 3.5 | −22.35 |
| 11 | 80 | 1 | 2.6 | 8.6 | −34.77 |
| 12 | 20 | 0.1 | 4.0 | — | — |

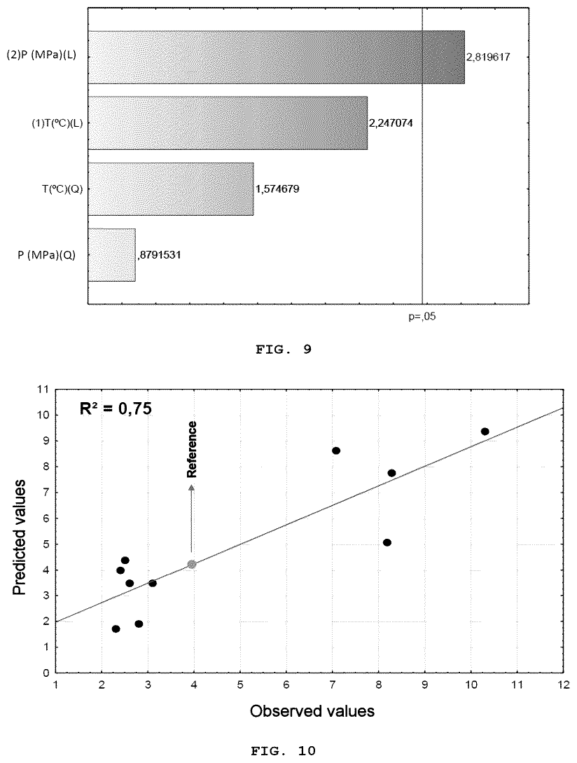

Data modeling generated an acceptable correlation between measured and predicted values, R²: 0.75 (FIG. 9), with pressure being a significant factor in explaining the results. Thus, and as can be seen in the aforementioned table, experiments with pressure equal to or greater than 0.5 MPa (experiments 3, 4, 6 and 8) showed increased migration; however, there is interaction (synergism) with temperature, since experiments such as 11 did not result in the same pattern observed.

The experiments were repeated with higher residual values, but this did not increase the correlation value beyond that already indicated, and FIG. 10 shows the response surface of the model. Although the significant effect exerted by pressure was linear, the quadratic curvature of the graph indicates that there is synergy between the factors and that temperature has relevance to the equilibrium. The extent of the effect, on the other hand, is lower than the effect observed for other parameters, although it cannot be overlooked precisely because it is a species with a longer carbon chain and, therefore, it is considered to be representative of the acidic species usually found (and reported in the literature) for produced water systems.

### Example 3: Implementation of Tests in Pressurized Systems with CO₂ in the Presence and Absence of Alkalinity

In the initial tests, 100 mL of heptol and 300 mL of water (with or without salinity) were used, with the addition of acidic species in the organic phase, in order to verify migration to the aqueous phase. As a rule, the system was kept under agitation at 500 rpm for 8 min, followed by rest for 10 min and, finally, collection of 50.0 mL aliquots of the aqueous phase for titrimetric analysis.

A 316 stainless steel alloy reactor (Parr®) (capacity for 600 mL and operational up to 20 MPa pressure) was used. For this purpose, a temperature range between 25° C. and 80° C. and pressure between 0.1 MPa and 1.0 MPa were used, and the reactor was pressurized with CO₂. Thus, the results were compared (in terms of migration of the acidic species (%, mol) by means of back titrations) and the influence of pressure and temperature on the partition was estimated, as well as possible synergistic effects with the sources of alkalinity added to the aqueous phase.

The partition equilibrium established by acidic species at the interface between the aqueous and oily phases is the result of the sum of effects (physical and chemical) that occur in this system, in a macroscopic and microscopic environment. The objective of the present experiment is to qualify and quantify the tendency of disturbance of the equilibrium generated by the presence of alkalinity in the aqueous phase. For this purpose, the species NaHCO₃ and Na₂CO₃ were used as alkalinity sources.

Previously, the back titration procedure was internalized and optimized (Example 1), ensuring that the reproducibility of the measurements was adequate (standard deviation values below 10%, for example) and that the recoveries were close to what was expected based on the purity of the reagents (i.e., close to or above 90%). Once these conditions were established, “control” experiments were performed in order to compare the results with each other and, thus, provide an overall view of the effect of each parameter and the possible synergies between the variables temperature, pressure (generated by CO₂) and alkalinity (NaHCO₃ and Na₂CO₃). Control experiments were performed (measuring the availability of the species in an aqueous medium, without using the reactor) by solubilizing the organic acid directly in the aqueous phase and titrating a 10.0 mL aliquot; the same way, experiments were performed in the reactor based on the standardization of heptol and salinity already discussed (3.5%). Some acids were difficult to solubilize, such as cyclohexanepentanoic acid and dicyclohexylacetic acid, requiring heating and 30 min in ultrasound to eliminate deposits and ensure the reproducibility of the tests.

Thus, the experiments were performed with previously standardized reagents, and with titration replicates, allowing a broad view of the partition equilibrium for each species. For the results with benzoic acid (Table 8), the availability of the acidic species is between 72.2 and 83.1 (%, mol) in the presence of NaHCO₃ (concentration of 3.45×10⁻⁵) and NaHCO₃+Na₂CO₃ (concentration of 3.48×10⁻⁵), respectively. In the reactor tests, in turn, the tendency is for a decrease in the migration of benzoic acid, since only 53.6 and 49.3 (%, mol) of the total added heptol was quantified in the aqueous phase. This result was verified by replicas and indicates that the parameter “alkalinity” significantly affects the partition equilibrium (decreasing the migration tendency to the aqueous phase).

Additionally, the migration to the aqueous phase was equally affected by the pressure in the reactor, tests with 1.0 MPa of CO₂, since the values were below the reference (82.0±3.0%, mol). However, the tests with alkalinity (and 1.0 MPa of pressure) resulted in the inversion of the trend, since an increase in the migration of benzoic acid was observed in relation to the reference values. The extent of the synergistic effect appears to not only compensate for the decrease in migration verified by tests carried out with the parameters (alkalinity, pressure and temperature) separately, but also increases migration to the aqueous phase to values between 93.3 and 100% (%, mol) at 25° C. and 88.9 and 88.3 (%, mol) at 80° C.

TABLE 8
Results for the partition equilibrium study with benzoic acid

| | Migration (% mol) |
|---|---|
| Reference (heptol + 3.5% NaCl) | 82.0 |
| 25° C. and 1.0 MPa | 64.4 |
| 80° C. and 1.0 MPa | 59.3 |

| | NaHCO₃ | Na₂CO₃ + NaHCO₃ |
|---|---|---|
| Availability (reference value) | 83.1 | 72.2 |
| 25° C. and 0.1 MPa | 53.6 | 49.3 |
| 25° C. and 1.0 MPa | 93.3 | 100.0 |
| 80° C. and 1.0 MPa | 88.6 | 88.3 |

| | |
|---|---|
| Standard Deviation | ±3.0 (% mol) |

For the study with pentanoic acid (Table 9), although the test values are different from those obtained with benzoic acid, the migration trends are similar since the reduction in the migration of the acidic species in the tests with only alkalinity was countered by the increase in migration in the tests with 1.0 MPa/25° C. (100 and 100%, mol) and 1.0 MPa/80° C. (96.3 and 79.9%, mol). The presence of pressure in the model system increases the migration of pentanoic acid to the aqueous phase and, in this case, generated migrations with values greater than 100%. These values are interpreted as being a consequence of the partial solubilization of CO₂ in the aqueous phase, given the experimental quantification that, at 25° C., up to 8.2×10⁻⁵ moles of carbonic acid were present in the medium; and at 80° C., 4.1×10⁻⁵ moles were present in the aqueous phase.

TABLE 9
Results for the partition equilibrium study with pentanoic acid

| | Migration (% mol) |
|---|---|
| Reference (heptol + 3.5% NaCl) | 77.0 |
| 25° C. and 1.0 MPa | 100 |
| 80° C. and 1.0 MPa | 82.4 |

| | NaHCO₃ | Na₂CO₃ + NaHCO₃ |
|---|---|---|
| Availability (reference value) | 90.7 | 66.0 |
| 25° C. and 0.1 MPa | 69.2 | 61.3 |
| 25° C. and 1.0 MPa | 100.0 | 100.0 |
| 80° C. and 1.0 MPa | 96.3 | 79.9 |

| | |
|---|---|
| Standard Deviation | ±6.0 (% mol) |

On the other hand, the same study performed with cyclohexanepentanoic acid (Table 10) resulted in significant increases in the migration tendency to the aqueous phase in the presence of alkalinity (100 and 100%, mol). Considering that the reference value is only 4.9 (%, mol), the extent of the effect is greater and in the opposite direction to that observed for benzoic and pentanoic acids. Furthermore, the migration results are also higher (82.7 and 71.1%, mol) while the simple imposition of temperature and pressure did not generate a gain in migration to the aqueous phase, and the presence of the acidic species in this case was not quantifiable (the quantification limit is approximately 1.5%).

TABLE 10
Results for the partition equilibrium
study with cyclohexanepentanoic acid

| | Migration (% mol) |
|---|---|
| Reference (heptol + 3.5% NaCl) | 4.9 |
| 25° C. and 1.0 MPa | ND |
| 80° C. and 1.0 MPa | ND |

| | NaHCO₃ | Na₂CO₃ + NaHCO₃ |
|---|---|---|
| Availability (reference value) | 100.0 | 100.0 |
| 25° C. and 0.1 MPa | 82.7 | 71.1 |
| 25° C. and 1.0 MPa | 100.0 | 100.0 |
| 80° C. and 1.0 MPa | 100.0 | 100.0 |

| | |
|---|---|
| Standard Deviation | ±1.3 (% mol) |

Again, the synergistic effect established between alkalinity and pressure/temperature can be observed, since the values always tended to 100% migration for the aqueous phase. Even so, the trend is clear for increased migration of cyclohexanepentanoic acid and with an extension much higher than that of the other acidic species. This result, in fact, allows to see that the structure of the acidic species affects the partition equilibrium and, therefore, should be considered in data modeling studies, as well as bringing an important point of discussion about which acidic species are mostly affected and should be the focus of studies and field measurements.

Finally, the study with 5-phenylpentanoic acid (Table 11) also generated migration trends of 100% both in the presence of alkalinity individually and together with pressure/temperature, while only the pressure of 1.0 MPa did not generate quantifiable results for this acidic species in the aqueous phase. Considering that the occurrence of resonance in the cyclic portion of the molecule is the chemical difference between this acid and cyclohexanepentanoic acid, it is indicated that the size of the acidic species (i.e., number of carbons) is preponderant in explaining the experimental result.

TABLE 11
Results for the partition equilibrium
study with 5-phenylpentanoic acid

| | Migration (% mol) |
|---|---|
| Reference (heptol + 3.5% NaCl) | 10.3 |
| 25° C. and 1.0 MPa | ND |
| 80° C. and 1.0 MPa | ND |

| | NaHCO₃ | Na₂CO₃ + NaHCO₃ |
|---|---|---|
| Availability (reference value) | 100.0 | 100.0 |
| 25° C. and 0.1 MPa | 100.0 | 100.0 |
| 25° C. and 1.0 MPa | 100.0 | 100.0 |
| 80° C. and 1.0 MPa | 100.0 | 100.0 |

| | |
|---|---|
| Standard Deviation | ±4.0 (% mol) |

The effect of the parameters presented in this results report, individually and together, are not usual in the literature and the operation in a model system adds important operational control capacity and consequent generation of knowledge with reproducibility and reliability. For the study of the partition equilibrium of the four reported acidic species, in the presence of alkalinity, the production water mimicking system proved to be even more relevant, since it allowed comparing the effects between the acidic species, as well as showed that there are synergisms between the physical and chemical factors, not only increasing the migration tendency, but also reversing this tendency (in the case of benzoic acid).

An equally important consideration can be made about the species with the longest carbon chain (cyclohexanepentanoic acid and 5-phenylpentanoic acid), which have as a basic difference the occurrence of resonance in the cyclic portion. While the partition equilibrium in the aqueous phase is 4.3% for the first species, it rises to 10.3% for the second species (this one with resonance in the structure). Both values are low when compared with benzoic acid (82% migration to the aqueous phase) and pentanoic acid (77% migration); however, they invariably tend to 100% migration (to the aqueous phase) in reactor tests (addition in the heptol fraction), and especially when the tests are performed under pressure (1.0 MPa). Considering that all values equal to or greater than 100% recovery indicate maximum migration to the aqueous phase, alkalinity can be considered the main variable influencing the partition equilibrium of acidic species above 10 carbons (provided that they have at least one naphthenic portion).

### Example 4: Study of Partition Equilibrium Under the Influence of Salinity

The variation in salinity (and its effect on acidic species) is due to the large variation in salinity found between the portions of bottom-hole water, middle-well water and surface water. Thus, systems with salinity (i.e., NaCl) between 3.5 and 30% (m·v⁻¹) were tested in a glass reactor, and neutralization titration was performed (in triplicate analysis), as previously described. Furthermore, the experimental results were generated in triplicate, using 99% NaCl (Sigma-Aldrich) with five acidic species.

Tests were performed (in triplicate) for different increasing concentrations of NaCl, namely: 3.5, 11.0, 18.0, 26.0 and 30.0% (m·v⁻¹) in the presence of five acidic species (at a concentration of 0.03 M for benzoic acid and pentanoic acid and 0.01 M for cyclohexanepentanoic acid, 5-phenylpentanoic acid and dicyclohexylacetic acid).

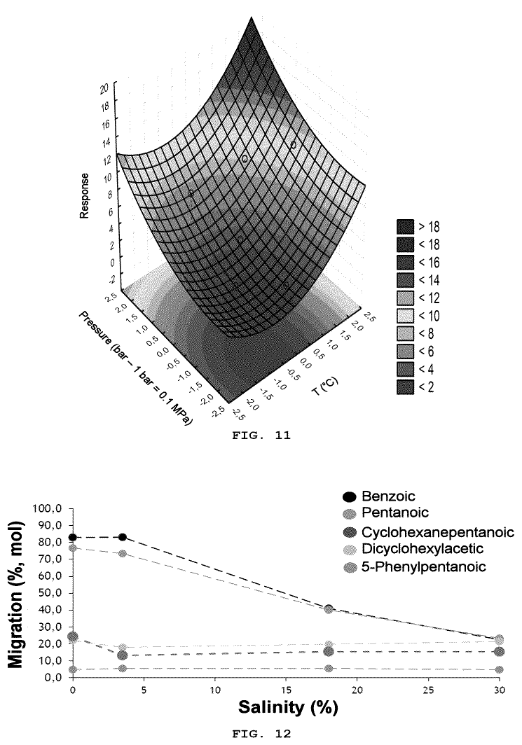

FIG. 12 summarizes the information, denoting the salting out effect of acidic species from the increasing addition of sodium chloride in the aqueous phase. However, due to the controlled conditions inherent to the model system, and considering that the study standardized the quantification of migration in terms of percentage of moles in the aqueous phase, it is possible to compare the extent of the effect in each of the five acidic species. Thus, in the case of benzoic acid, a reduction of approximately 400% in the number of moles contained in the aqueous phase is noted with the addition of sodium chloride (with 30% (m·v⁻¹), only 22.51% of benzoic acid remains in the aqueous phase).

With pentanoic acid, the salting out effect is approximately 300% between the tests without salinity and with 30% (m·v⁻¹) salinity. Additionally, the complementary experiment of this study, using 5-phenylpentanoic acid, resulted in the observation of the same salting out effect, although to a lesser extent than the others, reaching close to 38% with the addition of 30% NaCl (m·v⁻¹) in the aqueous phase. In the cases mentioned, except for 5-phenylpentanoic acid, the only addition of salinity with a non-significant effect (95% reliability) was the level of 3.5% (m·v⁻¹) of NaCl, in which there was no decrease in the migration of the acidic species.

The salting out effect appears to vary depending on the acidic species, since it has a different extent and is much more expressive in the two species with the greatest affinity for the aqueous fraction (and consequently with the shortest carbon chain). Table 12 presents the migration data for all acids studied in different concentrations of sodium chloride.

TABLE 12
Results of migration of acidic species
for different NaCl concentrations

| | Migration (%, mol) | | | | |
|---|---|---|---|---|---|
| | | | Cyclo- | | Dicyclo- |
| | | | hexane- | 5-Phenyl- | hexyl- |
| Salinity | Benzoic | Pentanoic | pentanoic | pentanoic | acetic |
| (%, m · m⁻¹) | Acid | Acid | Acid | Acid | Acid |
| 0 | 82.9 | 76.7 | 4.9 | 24.4 | 22.0 |
| 3.5 | 83.0 | 73.3 | 5.5 | 13.2 | 18.1 |
| 18.0 | 41.0 | 40.0 | 5.5 | 15.4 | 19.9 |
| 30.0 | 22.5 | 23.3 | 4.8 | 15.4 | 21.5 |

Additionally, for cyclohexanepentanoic acid, which establishes partition equilibria with reduced values in aqueous medium (close to 5%), a similar effect is observed to that observed for dicyclohexylacetic acid (with migration values around 20%). For both, there was no significant effect (95% reliability) on the addition of salinity to the medium. In short, it is understood that the increase in the polarizability of the medium (due to the presence of a greater quantity of ions in the system) did not affect the migration of these acidic species to the aqueous phase, which may be associated with the size of the chain and the spatial arrangement of the molecule, both of which are unfavorable to migration between phases (and which, therefore, appear to be more relevant for the partition equilibrium than the salinity of the medium).

On the other hand, comparing cyclohexanepentanoic and 5-phenylpentanoic acids, which differ chemically due to resonance in the cyclic portion of the carbon chain, it is noted that the effect of salinity was observed precisely for the acidic species with resonance. Thus, despite the chemical chain being longer than that of pentanoic and benzoic acids, the increase in ions in the aqueous phase made this portion energetically unfavorable, thus maintaining most of the 5-phenylpentanoic acid molecules in the organic portion (for which the resonant portion of the chain has precisely the greatest affinity).

### Example 5: Study of Partition Equilibrium Under the Influence of the Addition of Calcium and Strontium Salts to the Reaction System

The addition of salts to the reaction system is of fundamental importance when one intends to mimic field conditions. In this sense, in addition to continuing the study of the salinity effect (Example 4), the insertion of salts containing Ca²⁺ and Sr²⁺ ions, known to be present in seawater, was tested. To this end, the projects were based on the literature (Zheng, J. et al. Offshore produced water management: A review of current practice and challenges in harsh/Arctic environments. Mar. Pol. Bul. 2016, 104, 7-19) and four concentrations of each of the two ions were evaluated (400.0, 1200.0, 2600.0 and 9000.0 mg·L⁻¹), maintaining salinity at 3.5% (m·v⁻¹).

In short, the experimental results were generated in triplicate, using 99% NaCl to add salinity to the aqueous fraction and PA calcium chloride and PA strontium chloride to add calcium and strontium, respectively. The experiments were performed with five acidic species (benzoic acid, pentanoic acid, cyclohexanepentanoic acid, 5-phenylpentanoic acid and dicyclohexylacetic acid), and also had control tests to confirm the absence of interference from the reagents (possible impurities) on the results of the experiments.

The addition of calcium and strontium to the reaction system, in the form of salts, aims at mimicking the real scenario in which both species are present (regardless of the salinity of the medium), especially in the well condition. This study aimed at understanding the effect (on the migration of acids to the organic phase) of this ion in the system also at concentrations higher than 10 mM. Finally, the experiments (in triplicate) with divalent strontium ions followed the same rationale.

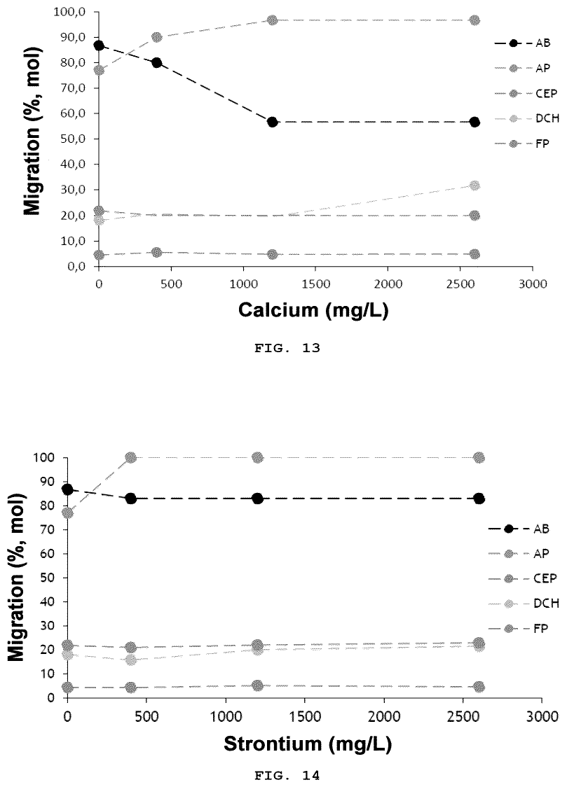

Considering that all experiments were performed under constant salinity conditions (3.5%, m·v⁻¹), it is relevant to note that it was possible to verify effects in different extents and directions among the acidic species (FIG. 13). Thus, for benzoic acid, the occurrence of a salting out effect was systematically observed in the experiments. The effect had an observable extent greater than 30%, indicating the relevance of this ion for the migration of this acid toward the aqueous phase, especially if it is considered that the levels observed are lower than the concentrations verified in the case of high salinities (30%, m·v⁻¹), even if the maximum addition is 2.6% (m·v⁻¹). The migration decreases from the concentration of 400 mg·L⁻¹ and remains constant even at higher concentrations (up to 9000 mg·L⁻¹). The decrease in the concentration of these species appears to follow the logic observed with the addition of sodium (behavior similar to the salinity experiment, see Example 4), and may correspond to the ionic effect of the organic phase, that is, it is independent of the nature of the ion itself.

In the case of experiments with pentanoic acid in the presence of calcium, the effect maintained a similar scale of magnitude, but in the opposite direction, even increasing the presence of this acidic species to levels close to 100% of the theoretical concentration (at a concentration of 400 mg·L⁻¹ of calcium) and maintaining this behavior even at higher concentrations. In short, this effect may be related to the formation of complexes with this divalent ion and, therefore, corroborating studies that report the effect of calcium in an aqueous medium (either through the formation of naphthenates or through the eventual greater affinity in relation to the sodium cation, which is more abundant), increasing the migration of species that associate with this ion.

For cyclohexanepentanoic acid, it is observed that in the presence of calcium ion there is no variation in migration to the aqueous phase, maintaining the behavior even at the maximum study concentrations (there was no significant difference between the ranges of consideration within a 95% confidence interval).

For dicyclohexylacetic acid, at a concentration of 2600 mg·L⁻¹ of calcium, migration increases from 22 to 31% (m·v⁻¹). For 5-phenylpentanoic acid, there is no variation in migration in the presence of calcium ion, regardless of the ion concentration. Comparing 5-phenylpentanoic acid and cyclohexanepentanoic acid (distinct only by the occurrence of resonance in the former) and considering that resonance is a stabilizing effect of the molecular structure, there is possibly greater stability in the organic phase regardless of the presence of calcium in the medium (reinforcing that salinity generated a salting out effect), thus not causing deprotonation of the acid and consequent migration to the aqueous phase. Furthermore, it can be assumed that there is no energy gain arising from the formation of compounds such as calcium naphthenates, either due to the number of carbons or the structural conformation.

Table 13 summarizes the results observed for the experiments with the addition of calcium and strontium. The same experimental procedure was performed with strontium ions (FIG. 14) and resulted in observed effects distinct from those obtained with calcium. Here, 5-phenylpentanoic acid, dicyclohexylacetic acid and cyclohexanepentanoic acid (low tendency to migrate to the aqueous phase) presented similar results: no effect in the case of cyclohexanepentanoic and dicyclohexylacetic acids and decreased migration (with the same direction and extent) for 5-phenylpentanoic acid, at an ion concentration of 2600 mg·L⁻¹.

For the species of smaller size and volume, benzoic and pentanoic acids, the salting out and salting in effects (respectively) were similar, but to a different extent, since benzoic acid still migrated above 80% (m·v⁻¹) to the aqueous phase. Here, therefore, it may be that not only divalence is relevant for this effect, but also the size of the ion (strontium has a larger atomic radius than calcium), which corroborates the previous reasoning about the preferential formation of stable structures involving calcium ion and carboxylic groups.

Pentanoic acid in particular showed an increase in migration to the aqueous phase, even with only 400 mg·L⁻¹ of strontium in the aqueous phase. Assuming that this effect results from the decrease in energy in the deprotonated form (which migrates to the aqueous phase), it is understood that there are differences between the complexes formed with calcium and strontium (whether they are naphthenates or not), and this may generate considerable influence on the equilibrium of smaller species (with less than 7 carbons), and may even compensate for eventual salting out effects arising from the salinity of the aqueous phase.

TABLE 13
Titration results involving experiments with addition
of calcium and strontium in the aqueous phase

| | Migration (%, mol) | | | | | | |
|---|---|---|---|---|---|---|---|
| | Absence | Calcium (mg · L⁻¹) | | | Strontium (mg · L⁻¹) | | |
| Acidic Species | of ions | 400 | 1200 | 2600 | 400 | 1200 | 2600 |
| Benzoic Acid | 82.0 | 80.0 | 56.7 | 56.7 | 83.0 | 83.0 | 83.0 |
| Pentanoic Acid | 77.0 | 90.0 | 98.7 | 96.7 | 100.0 | 100.0 | 100.0 |
| Cyclohexanepentanoic | 4.9 | 5.5 | 4.7 | 4.8 | 4.4 | 5.3 | 4.8 |
| Acid | | | | | | | |
| Dicyclohexylacetic | 22.1 | 20.7 | 19.6 | 31.8 | 15.8 | 20.0 | 21.5 |
| Acid | | | | | | | |
| 5-Phenylpentanoic | 10.4 | 5.5 | 4.7 | 3.4 | 4.4 | 5.3 | 4.8 |
| Acid | | | | | | | |

### Example 6: Study of Partition Equilibrium Under the Influence of the Presence of Demulsifiers and Surfactants in the Reaction System

The presence of chemical compounds usually added in the chain of unit operations of oil extraction and production, such as demulsifiers and surfactants, can affect the partition equilibrium of species in this water/oil system. In order to understand the extent of this effect, as well as discern the relevance of the chemical structure on this effect, the following items were chosen:
Dissolvan 961 (Clariant), composed of oil hydrocarbons (CAS 918-811-1) and ethanol (CAS 64-17-5);
Dissolvan 974 (Clariant), composed of EO-PO block polymer poly adducts and Bisphenol-A-diglycidyl ether (CAS 68123-18-2) and ethanol (CAS 64-17-5);
Tetrabutylammonium chloride (CAS 1112-67-0), 97% purity;
Cetyltrimethylammonium chloride (CAS 112-02-7), 25% in water; and
Trimethylammonium chloride (CAS 593-81-7), 98% purity.

For this study, the experiments (in triplicate) used between 100.0 and 1000.0 mg·L⁻¹ of each compound, comparing the results with control tests (absence of the component), all with constant salinity at 3.5% (m·v⁻¹). Finally, neutralization titration with a standardized NaOH titrant solution was used to determine the effect on the partition equilibrium for four acidic species (the same as in the other examples, except for dicyclohexylacetic acid).

To study the partition equilibrium between the acidic species in the presence of two demulsifiers, experiments were performed to evaluate the eventual effects on the partition equilibrium of benzoic acid (0.03 M), in relation to control experiments, as shown in Table 14.

In short, comparing the migration of benzoic acid with the other results using this species in a medium with salinity (3.5%, m·v⁻¹) and room pressure and temperature, a decrease in the migration tendency to the aqueous phase (always below 71%) is noted (FIG. 15). This salting out effect has a smaller extent than that observed in the presence of calcium, and much lower than that observed with the addition of salinity in the aqueous phase; however, it is still observable and occurs even in the presence of only 50 mg·L⁻¹ of demulsifiers (both), that is, in concentrations much lower than those used for the other parameters under study.

Pentanoic acid, on the other hand, had no observable effect (with 95% significance), even when faced with demulsifier concentrations of 1000 mg·L⁻¹. While a salting in effect was observed in the presence of calcium and strontium, and a salting out effect in the presence of increasing salinity in the aqueous phase, here the partition equilibrium remained between 67 and 77.4%. Considering that the maximum study point already implies the occurrence of a demulsifier addition peak that is difficult to reach in the field, it is understood that this effect can be neglected for species with a shorter carbon chain (<7 carbons) of an aliphatic nature.

Considering that demulsifiers are added to ensure the destabilization of emulsions observed in the oil and gas segment, this is a class of compounds usually formed by poly(ethylene oxide-b-propylene oxide) copolymers and are normally formulated from a mixture of demulsifier bases, dispersed in organic solvents, usually aromatic solvents and alcohols (Amaravathi, M.; Pandey. Ethylene oxide-propylene oxide copolymer as demulsifier for petroleum emulsion. Research and Industry, 1991, 36, 198-202; Rowan, B. The use of chemicals in oilfield demulsification. Industrial Applications of Surfactants III, 1992, 242-251). Although its mechanism of action is not fully elucidated, it is understood that its molecules are adsorbed in the aqueous fraction and, therefore, they facilitate the removal of natural emulsifiers from this matrix, which facilitates the coalescence of the droplets (Shu, G. et al. Evaluation of newly developed reverse demulsifiers and cationic polyacrylamide flocculants for efficient treatment of oily produced water. Colloids and Surfaces A: Physicochemical and Engineering Aspects, 2021, 610, 125646; Deng, S. et al. Destabilization of oil droplets in produced water from ASP flooding, Colloids Surf. A Physicochem. Eng. Asp. 2005, 252, 113-119; Urdhal, O.; Movik, A. E.; Sjoblom, J. Water-in-crude oil emulsion from Norwegian continental shelf 8. Surfactant and macromolecular destabilization. Colloids and Surface A: Physicochemical and Engineering Aspects, 1993, 74, 293-302). Here, the study with the acidic species with the longest carbon chain, in the presence of this class of compounds, resulted in partition equilibria with a significant effect and toward the aqueous phase, especially with the addition of 1000 mg·L⁻¹. In the case of cyclohexanepentanoic acid, the effect deserves to be highlighted because it resulted in an increase close to 100%, precisely with this acidic species that did not show an effect due to the action of divalent ions and salinity.

As for the study with 5-phenylpentanoic acid, a resonance structure, the effect was observed even with 50 mg·L⁻¹ of D961 (increase of approximately 100% in migration to the aqueous phase). Although the composition of this demulsifier is not known, the salting in effect, opposite to the patterns observed in the presence of divalent ion salinity, can be attributed to the presence of aromatic hydrocarbons that naturally have chemical affinity with this acidic species.

TABLE 14
Results of the neutralization titrations of the study with addition
of demulsifier to the system containing benzoic acid, pentanoic
acid, cyclohexanepentanoic acid and 5-phenylpentanoic acid

| | Migration (%, mol) | | | | | | |
|---|---|---|---|---|---|---|---|
| | Absence | D961 (mg · L⁻¹) | | | D974 (mg · L⁻¹) | | |
| Acidic Species | of ions | 50 | 100 | 1000 | 50 | 100 | 1000 |
| Benzoic Acid | 82.0 | 62.3 | 70.3 | 68.4 | 63.1 | 57.3 | 59.7 |
| Pentanoic Acid | 77.0 | 67.0 | 70.0 | 75.7 | 66.9 | 74.3 | 74.8 |
| Cyclohexanepentanoic | 4.9 | 5.42 | 5.16 | 10.2 | 4.78 | 5.26 | 12.80 |
| Acid | | | | | | | |
| 5-Phenylpentanoic | 10.4 | 21.0 | 21.0 | 23.0 | 18.0 | 16.0 | 14.6 |
| Acid | | | | | | | |

Considering the study with surfactants, the (individual) addition of three species to the control system was evaluated (tetrabutylammonium chloride (TBA), tetramethylammonium chloride (TMA) and cetyltrimethylammonium chloride (CTA)). For this purpose, the surfactants have distinct chemical structures, namely the size of the side chain, and their eventual effect on the partition equilibrium of the acidic species was evaluated.

Surfactants composed of the chemical group of quaternary ammonium salts are organic compounds that have an amphiphilic behavior, that is, they interact with polar and apolar substances. The apolar (hydrophobic) portion is usually a hydrocarbon chain, while the polar (hydrophilic) part can be ionic (anionic or cationic), non-ionic or amphoteric. This property makes them versatile in industrial applications and, of course, useful for use as surfactants and surface tension reducing agents in water/oil systems.

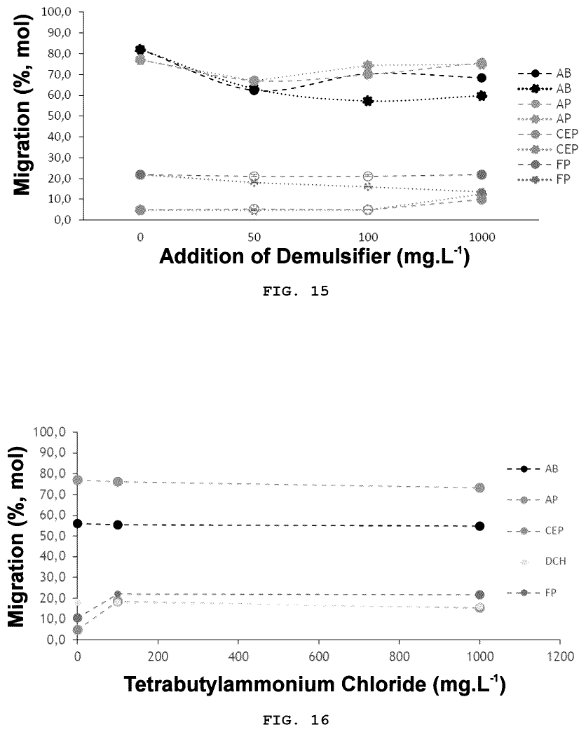

The results of the experiments performed with the acidic species, in the presence of the surfactant TBA, are shown in FIG. 16. The presence of this compound, even at different concentrations, had no significant effect on the migration of benzoic and pentanoic acids to the aqueous phase. On the other hand, the same experiments performed for cyclohexanepentanoic and 5-phenylpentanoic acids indicated an increase in the tendency to migrate to the aqueous phase, resulting in an increase of 370% for the first and 100% for the second. Considering that the effect is already noticeable with 100 mg·L⁻¹ of surfactant, there is a clear distinction in the effect between species with shorter/longer carbon chains, as well as in the presence of resonance (which again decreases the affinity of the acidic species for the aqueous phase, attenuating the effect of TBA on the partition equilibrium), in the case of 5-phenylpentanoic acid.

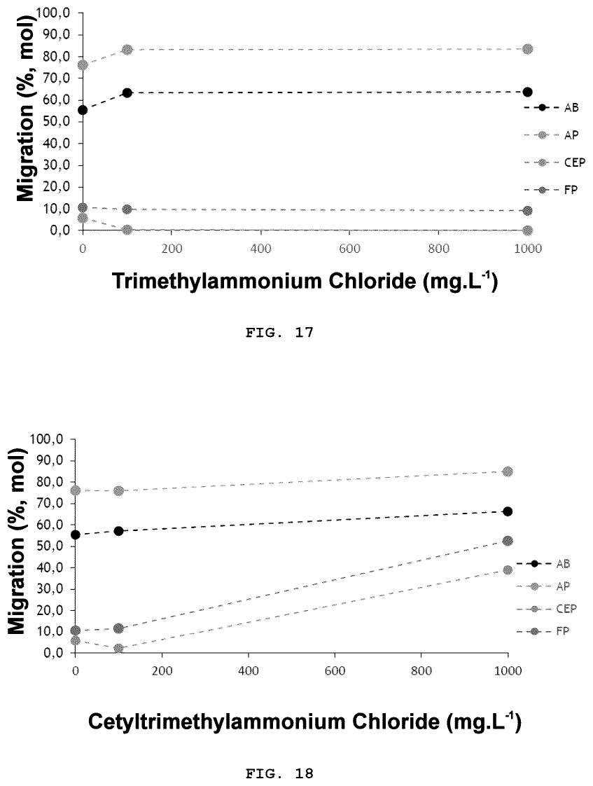

FIG. 17 presents the migration data of the acidic species in the presence of the surfactant TMA. In this case, the results indicate an observable effect on the partition equilibrium of benzoic and pentanoic acids, with an increase in the affinity for the hydrophilic phase upon addition of the surfactant to the system (effect of approximately 10%). For the acids with a longer carbon chain, there was no observable effect for 5-phenylpentanoic acid (with 95% reliability), while cyclohexanepentanoic acid had migration tending to zero (reference value at 4.9%), in clear opposition to the effect of the surfactant TBA. There were no significant changes in migration regardless of the presence of that compound. The structure of this surfactant resembles the first, already discussed, but with a shorter alkyl chain, which may explain the absence of effect for all acidic species.

Finally, the same experimental logic was applied to the surfactant CTA (FIG. 18), and similar results were obtained regarding the displacement of equilibrium to the aqueous phase for both benzoic acid and pentanoic acid, since the increase in migration to this phase was close to 10% (in moles). On the other hand, in this case, the effect became observable only with the addition of 1000 mg·L⁻¹, while TMA resulted in this same effect with 100 mg·L⁻¹ in the aqueous phase.

Additionally, the acidic species with greater volume and area had their partition equilibria affected in favor of the hydrophilic phase, since cyclohexanepentanoic acid (above 700%) and 5-phenylpentanoic acid (close to 500%) migrated more easily to this phase in the presence of CTA. This significant effect is not paralleled in the experiments reported herein, since it resulted in migrations of 38.9 and 52.6% (in moles) of the acidic species, respectively (Table 15). Although this effect was observed only with the addition of 1000 mg·L⁻¹, the alkyl group of the hydrophobic chain of the surfactant, in this case with 16 carbons, appears to have a significant effect on this displacement of the equilibrium, generating compounds with lower surface tension. It is assumed that the formation of micelles containing the acidic species in question occurs.

Although the increase in migration to the aqueous phase can be predicted as a result of the formation of micelles in which the surfactant molecules act as carriers of the acidic species to the aqueous phase, it is noted that the hydrophobic chain of each surfactant affects the extent of this effect and should, therefore, be taken into account when considering its addition to acidic production waters. While the effect of TMA was more evident for the compounds with the shortest carbon chain (as it has the shortest alkyl chain of the three evaluated surfactants), the other surfactants had a greater effect precisely with the largest acidic species (again, according to the similarity of the alkyl chain of the surfactants). Thus, it follows that the observable effect is dependent on the target compounds, and on the affinity of the chemical structure of the surfactant with the acidic species present in the medium.

TABLE 15
Results of the neutralization titrations of the study with addition
of surfactant to the system containing acidic species

| | Migration (%, mol) | | | | | | |
|---|---|---|---|---|---|---|---|
| | Absence of | Surfactants (100 and 1000 mg · L⁻¹) | | | | | |
| Acidic Species | surfactant | TBA₁₀₀ | TBA₁₀₀₀ | CTA₁₀₀ | CTA₁₀₀₀ | TMA₁₀₀ | TMA₁₀₀₀ |
| Benzoic Acid | 82.0 | 55.5 | 54.8 | 57.1 | 66.3 | 63.4 | 63.7 |
| Pentanoic Acid | 77.0 | 76.2 | 73.3 | 76.0 | 85.0 | 83.2 | 83.5 |
| Cyclohexanepentanoic | 4.9 | 18.5 | 18.4 | 2.1 | 38.9 | 0.0 | 0.0 |
| Acid | | | | | | | |
| 5-Phenylpentanoic | 10.4 | 22.0 | 21.6 | 11.5 | 52.6 | 9.4 | 9.1 |
| Acid | | | | | | | |

TBA: tetrabutylammonium chloride; CTA: cetyltrimethylammonium chloride; TMA: trimethylammonium chloride

Furthermore, taking as a basis the extent of the effect on species with a longer carbon chain (resonant or not), this parameter has a more pronounced effect than that observed for demulsifying compounds, although it must be considered that the compounds were used herein without dilution and the presence of components (inert or not) inherent to the commercial formulations used in the case of demulsifiers D961 and D974.

1. A method for determining the partition of naphthenic acids in a system that mimics production water, characterized in that it comprises:
a) mixing, in a mimicking system, an aqueous phase and an oily phase comprising acidic species, varying parameters of pH, salinity, alkalinity as a function of carbonated species, concentration of complex-forming divalent ions, temperature and/or pressure, under mechanical agitation;
b) collecting samples of the aqueous fraction; and
c) performing titration of the collected aqueous phase samples.

2. The method according to claim 1, characterized in that step a) is carried out under a pressure ranging from 0.1 MPa to 1 MPa.

3. The method according to claim 2, characterized in that the pressure variation is controlled by means of the introduction of N₂ or CO₂.

4. The method according to claim 1, characterized in that step a) is carried out at a temperature equal to or up to 60° C. above room temperature.

5. The method according to claim 4, characterized in that step a) is carried out at a temperature of 25° C. to 80° C.

6. The method according to claim 1, characterized in that the ratio between the oily phase and the aqueous phase of the mixture in step a) is 3:1.

7. The method according to claim 1, characterized in that the oily phase in step a) comprises heptol (90% to 10% (m/m) heptane and 10% to 90% (m/m) toluene) or crude oil.

8. Method according to claim 7, characterized in that the oily phase of step a) comprises heptol composed of 70% (m/m) heptane and 30% (m/m) toluene.

9. The method according to claim 1, characterized in that the acidic components added to the oily phase are selected from benzoic acid, pentanoic acid, cyclohexanepentanoic acid, 5-phenylpentanoic acid, dicyclohexylacetic acid and combinations thereof.

10. The method according to claim 1, characterized in that the aqueous phase comprises ultrapure or distilled water.

11. The method according to claim 1, characterized in that step a) is carried out under mechanical agitation with rotations between 200 and 1000 rpm.

12. The method according to claim 11, characterized in that the mixture is kept under agitation for 2 to 30 minutes.

13. The method according to claim 1, characterized in that the samples obtained in step b) are immediately subjected to step c) or are kept under refrigeration for later analysis.

14. The method according to claim 1, characterized in that step b) further comprises obtaining samples of the oily phase.

15. The method according to claim 1, characterized in that step c) is potentiometric titration performed with a NaOH solution, optionally in an automatic titrator.

16. A production water mimicking system, characterized in that it comprises a mechanical agitation system; a resistant material reactor, comprising a single inlet for the aqueous phase, organic phase and sample; a gas inlet controller; a heating and temperature control system; and a pressure relief valve sample collection system.

17. The mimicking system according to claim 16, characterized in that the resistant material reactor has volumes between 100 and 600 mL.

18. The mimicking system according to claim 16, characterized in that the resistant material reactor is made of material resistant to corrosion by oxidative and/or ionic species.

19. The mimicking system according to claim 18, characterized in that the resistant material reactor is made of steel alloys or glass.

20. The mimicking system according to claim 16, characterized in that the gas inlet controller is a manual controller by means of a ball valve for ¼ stainless steel piping, optionally in which the valve is ⅜″ (0.9525 cm) or ½″ (1.27 cm).

21. The mimicking system according to claim 16, characterized in that the mechanical agitation system and the heating and temperature control system have the same controller, which is preferably a digital bivolt reaction controller.

22. The mimicking system according to claim 16, characterized in that the heating and temperature control system is an analog or digital controller, with a thermostatic bath.
